(19) United States
(12) Patent Application Publication
(10) Pub. No.: US 2004/0198740 A1
(43) Pub. Date: Oct. 7, 2004

(54) Therapies relating to combinations of aldose reductase inhibitors and cyclooxygenase-2

(75) Inventor: Banavara L. Mylari, Waterford, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/810,880

(22) Filed: Mar. 25, 2004

Related U.S. Application Data
(62) Division of application No. 10/137,472, filed on Apr. 30, 2002.
(60) Provisional application No. 60/287,524, filed on Apr. 30, 2001.

(51) Int. Cl. A61K 31/501

(52) U.S. Cl. 514/252.02; 514/252.03; 514/252.04; 514/252.05; 514/252.06

(57) ABSTRACT

This invention relates to pharmaceutical compositions and kits comprising pyridazinone compounds and cyclooxygenase-2 inhibitors, therapeutic methods of treatment or prevention of certain complications arising from diabetes mellitus in mammals and therapeutic methods of treatment or prevention of cardiac tissue ischemia in mammals.

### FIELD OF THE INVENTION

This invention relates to pharmaceutical compositions and kits comprising pyridazinone aldose reductase inhibitor compounds and cyclooxygenase-2 inhibitors, therapeutic methods of treatment or prevention of certain complications arising from diabetes mellitus in mammals and therapeutic methods of treatment or prevention of cardiac tissue ischemia in mammals.

### BACKGROUND OF THE INVENTION

The enzyme aldose reductase is involved in regulating the reduction of aldoses, such as glucose and galactose, to their corresponding polyols, such as sorbitol and galactitol. Sulfonyl pyridazinone compounds of formula I and formula II of this invention are useful as aldose reductase inhibitors in the treatment and prevention of diabetic complications of humans and other mammals associated with increased polyol levels in certain tissues (e.g., nerve, kidney, lens and retina tissue) of affected humans and other mammals.

French Patent Publication No. 2647676 discloses pyridazinone derivatives having substituted benzyl side chains and benzothiazole side chains as being inhibitors of aldose reductase.

U.S. Pat. No. 4,251,528 discloses various aromatic carbocyclic oxophthalazinyl acetic acid compounds, as possessing aldose reductase inhibitory properties.

Commonly assigned U.S. Pat. No. 4,939,140 discloses heterocyclic oxophthalazinyl acetic acid compounds.

Commonly assigned U.S. Pat. No. 4,996,204 discloses pyridopyridazinone acetic acid compounds useful as aldose reductase inhibitors.

U.S. Pat. No. 5,834,466 discloses a method for limiting or decreasing the extent of ischemic damage due to metabolic and ionic abnormalities of the heart tissue resulting from Ischemic insult by treatment with a compound such as an aldose reductase inhibitor which reduces NADH/NAD+ ratio and stimulates glycolysis to produce ATP.

### SUMMARY OF THE INVENTION

One aspect of this invention is pharmaceutical compositions comprising a first compound selected from: a compound of formula I

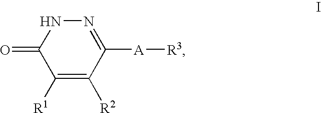

and a compound of formula II

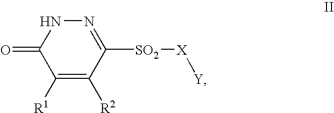

or a prodrug of said first compound, or a pharmaceutically acceptable salt of said first compound or said prodrug,

wherein:

A is S, SO or SO₂;

R¹ and R² are each independently hydrogen or methyl;

R³ is Het¹, —CHR⁴Het¹ or NR⁶R⁷;

R⁴ is hydrogen or (C₁-C₃)alkyl;

R⁶ is (C₁-C₆)alkyl, aryl or Het²;

R⁷ is Het³;

Het¹ is pyridyl, pyrimidyl, pyrazinyl, pyridazinyl, quinolyl, isoquinolyl, quinazolyl, quinoxalyl, phthalazinyl, cinnolinyl, naphthyridinyl, pteridinyl, pyrazinopyrazinyl, pyrazinopyridazinyl, pyrimidopyridazinyl, pyrimidopyrimidyl, pyridopyrimidyl, pyridopyrazinyl, pyridopyridazinyl, pyrrolyl, furanyl, thienyl, imidazolyl, oxazolyl, thiazolyl, pyrazolyl, isoxazolyl, isothiazolyl, triazolyl, oxadiazolyl, thiadiazolyl, tetrazolyl, indolyl, benzofuranyl, benzothienyl, benzimidazolyl, benzoxazolyl, benzothiazolyl, indazolyl, benzisoxazolyl, benzisothiazolyl, pyrrolopyridyl, furopyridyl, thienopyridyl, imidazolopyridyl, oxazolopyridyl, thiazolopyridyl, pyrazolopyridyl, isoxazolopyridyl, isothiazolopyridyl, pyrrolopyrimidyl, furopyrimidyl, thienopyrimidyl, imidazolopyrimidyl, oxazolopyrimidyl, thiazolopyrimidyl, pyrazolopyrimidyl, isoxazolopyrimidyl, isothiazolopyrimidyl, pyrrolopyrazinyl, furopyrazinyl, thienopyrazinyl, imidazolopyrazinyl, oxazolopyrazinyl, thiazolopyrazinyl, pyrazolopyrazinyl, isoxazolopyrazinyl, isothiazolopyrazinyl, pyrrolopyridazinyl, furopyridazinyl, thienopyridazinyl, imidazolopyridazinyl, oxazolopyridazinyl, thiazolopyridazinyl, pyrazolopyridazinyl, isoxazolopyridazinyl or isothiazolopyridazinyl; Het¹ is independently optionally substituted with up to a total of four substituents independently selected from R⁸, R⁹, R¹⁰ and R¹¹; wherein R⁸, R⁹, R¹⁰ and R¹¹ are each taken separately and are each independently halo, formyl, (C₁-C₆)alkoxycarbonyl, (C₁-C₆)alkylenyloxycarbonyl, (C₁-C₄)alkoxy-(C₁-C₄)alkyl, C(OH)R¹²R¹³, (C₁-C₄)alkylcarbonylamido, (C₃-C₇)cycloalkylcarbonylamido, phenylcarbonylamido, phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, (C₁-C₄)alkylsulfenyl, (C₁-C₄)alkylsulfonyl, (C₃-C₇)cycloalkyl, (C₁-C₄)alkyl optionally substituted with up to three fluoro or (C₁-C₄)alkoxy optionally substituted with up to five fluoro; said phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, in the definition of R⁸, R⁹, R¹⁰ and R¹¹ are optionally substituted with up to three substituents independently selected from hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said imidazolyl, oxazolyl, isoxazolyl, thiazolyl and pyrazolyl in the definition of R⁸, R⁹, R¹⁰ and R¹¹ are optionally substituted with up to two substituents independently selected from hydroxy, halo, C₁-C₄)alkyl, hydroxy-(C₁-C₄)alkyl, (C₁-C₄)alkoxy-(C₁-C₄)alkyl, C₁-C₄)alkyl-phenyl optionally substituted in the phenyl portion with one Cl, Br, OMe, Me or SO₂-phenyl wherein said SO₂-phenyl is optionally substituted in the phenyl portion with one Cl, Br, OMe, Me, (C₁-C₄)alkyl optionally substituted with up to five fluoro, or (C₁-C₄)alkoxy optionally substituted with up to three fluoro; R¹² and R¹³ are each independently hydrogen or (C₁-C₄)alkyl;

Het² and Het³ are each independently imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy; Het² and Het³ are each independently optionally substituted with up to a total of four substituents independently selected from R¹⁴, R¹⁵, R¹⁶ and R¹⁷, wherein R¹⁴, R¹⁵, R¹⁶ and R¹⁷ are each taken separately and are each independently halo, formyl, (C₁-C₆)alkoxycarbonyl, (C₁-C₆)alkylenyloxycarbonyl, (C₁-C₄)alkoxy-(C₁-C₄)alkyl, C(OH)R¹⁸R¹⁹, (C₁-C₄)alkylcarbonylamido, (C₃-C₇)cycloalkylcarbonylamido, phenylcarbonylamido, phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, (C₁-C₄)alkylsulfenyl, (C₁-C₄)alkylsulfonyl, (C₃-C₇)cycloalkyl, (C₁-C₄)alkyl optionally substituted with up to three fluoro or (C₁-C₄)alkoxy optionally substituted with up to five fluoro; said phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, in the definition of R¹⁴, R¹⁵, R¹⁶ and R¹⁷ are optionally substituted with up to three substituents independently selected from hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said imidazolyl, oxazolyl, isoxazolyl, thiazolyl and pyrazolyl in the definition of R¹⁴, R¹⁵, R¹⁶ and R¹⁷ are optionally substituted with up to two substituents independently selected from hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to three fluoro; and R¹⁸ and R¹⁹ are each independently hydrogen or (C₁-C₄)alkyl;

X and Y together are CH₂—CH(OH)—Ar or CH₂—C(O)—Ar, or

X is a covalent bond, NR²⁰ or CHR²¹, wherein, R²⁰ is (C₁-C₃)alkyl or a phenyl that is optionally substituted with one or more substituents selected from OH, F, Cl, Br, I, CN, CF₃, (C₁-C₆)alkyl, O—(C₁-C₆)alkyl, S(O)ₙ—(C₁-C₆)alkyl and SO₂—NR²²R²³, and R²¹ is hydrogen or methyl, and

Y is a phenyl or naphthyl ring optionally substituted with one or more substituents selected from Ar, OH, F, Cl, Br, I, CN, CF₃, (C₁-C₆)alkyl, O—(C₁-C₆)alkyl, S(O)ₙ—(C₁-C₆)alkyl and SO₂—NR²²R²³;

Ar is a phenyl or naphthyl ring optionally substituted with one or more substituents selected from F, Cl, Br, I, CN, CF₃, (C₁-C₆)alkyl, O—(C₁-C₆)alkyl, S(O)ₙ—(C₁-C₆)alkyl and SO₂—NR²²R²³;

n is independently for each occurrence 0, 1 or 2;

R²² is independently for each occurrence H, (C₁-C₆)alkyl, phenyl or naphthyl; and

R²³ is independently for each occurrence (C₁-C₆)alkyl, phenyl or naphthyl,

provided that when R³ is NR⁶R⁷, then A is SO₂, and a second compound that is a cyclooxygenase-2 inhibitor, a prodrug of said second compound or a pharmaceutically acceptable salt of said second compound or said prodrug.

Another aspect of this invention is kits comprising:

a first dosage form comprising a first compound selected from:

a compound of formula I

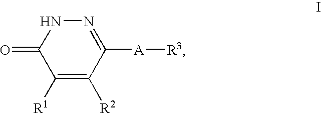

and a compound of formula II

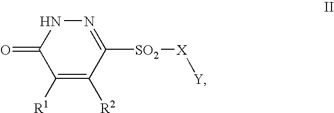

or a prodrug of said first compound, or a pharmaceutically acceptable salt of said first compound or said prodrug,

wherein:

A is S, SO or SO₂;

R¹ and R² are each independently hydrogen or methyl;

R³ is Het¹, —CHR⁴Het¹ or NR⁶R⁷;

R⁴ is hydrogen or (C₁-C₃)alkyl;

R⁶ is (C₁-C₆)alkyl, aryl or Het²;

R⁷ is Het³;

Het¹ is pyridyl, pyrimidyl, pyrazinyl, pyridazinyl, quinolyl, isoquinolyl, quinazolyl, quinoxalyl, phthalazinyl, cinnolinyl, naphthyridinyl, pteridinyl, pyrazinopyrazinyl, pyrazinopyridazinyl, pyrimidopyridazinyl, pyrimidopyrimidyl, pyridopyrimidyl, pyridopyrazinyl, pyridopyridazinyl, pyrrolyl, furanyl, thienyl, imidazolyl, oxazolyl, thiazolyl, pyrazolyl, isoxazolyl, isothiazolyl, triazolyl, oxadiazolyl, thiadiazolyl, tetrazolyl, indolyl, benzofuranyl, benzothienyl, benzimidazolyl, benzoxazolyl, benzothiazolyl, indazolyl, benzisoxazolyl, benzisothiazolyl, pyrrolopyridyl, furopyridyl, thienopyridyl, imidazolopyridyl, oxazolopyridyl, thiazolopyridyl, pyrazolopyridyl, isoxazolopyridyl, isothiazolopyridyl, pyrrolopyrimidyl, furopyrimidyl, thienopyrimidyl, imidazolopyrimidyl, oxazolopyrimidyl, thiazolopyrimidyl, pyrazolopyrimidyl, isoxazolopyrimidyl, isothiazolopyrimidyl, pyrrolopyrazinyl, furopyrazinyl, thienopyrazinyl, imidazolopyrazinyl, oxazolopyrazinyl, thiazolopyrazinyl, pyrazolopyrazinyl, isoxazolopyrazinyl, isothiazolopyrazinyl, pyrrolopyridazinyl, furopyridazinyl, thienopyridazinyl, imidazolopyridazinyl, oxazolopyridazinyl, thiazolopyridazinyl, pyrazolopyridazinyl, isoxazolopyridazinyl or isothiazolopyridazinyl; Het¹ is independently optionally substituted with up to a total of four substituents independently selected from R⁸, R⁹, R¹⁰ and R¹¹; wherein R⁸, R⁹, R¹⁰ and R¹¹ are each taken separately and are each independently halo, formyl, (C₁-C₆)alkoxycarbonyl, (C₁-C₆)alkylenyloxycarbonyl, (C₁-C₄)alkoxy-(C₁-C₄)alkyl, C(OH)R¹²R¹³, (C₁-C₄)al kylcarbonylamido, (C₃-C₇)cycloalkylcarbonylamido, phenylcarbonylamido, phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, (C₁-C₄)alkylsulfenyl, (C₁-C₄)alkylsulfonyl, (C₃-C₇)cycloalkyl, (C₁-C₄)alkyl optionally substituted with up to three fluoro or (C₁-C₄)alkoxy optionally substituted with up to five fluoro; said phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, in the definition of R⁸, R⁹, R¹⁰ and R¹¹ are optionally substituted with up to three substituents independently selected from hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said imidazolyl, oxazolyl, isoxazolyl, thiazolyl and pyrazolyl in the definition of R⁸, R⁹, R¹⁰ and R¹¹ are optionally substituted with up to two substituents independently selected from hydroxy, halo, C₁-C₄)alkyl, hydroxy-(C₁-C₄)alkyl, (C₁-C₄)alkoxy-(C₁-C₄)alkyl, C₁-C₄)alkyl-phenyl optionally substituted in the phenyl portion with one Cl, Br, OMe, Me or SO₂-phenyl wherein said SO₂-phenyl is optionally substituted in the phenyl portion with one Cl, Br, OMe, Me, (C₁-C₄)alkyl optionally substituted with up to five fluoro, or (C₁-C₄)alkoxy optionally substituted with up to three fluoro; R¹² and R¹³ are each independently hydrogen or (C₁-C₄)alkyl;

Het² and Het³ are each independently imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy; Het² and Het³ are each independently optionally substituted with up to a total of four substituents independently selected from R¹⁴, R¹⁵, R¹⁶ and R¹⁷, wherein R¹⁴, R¹⁵, R¹⁶ and R¹⁷ are each taken separately and are each independently halo, formyl, (C₁-C₆)alkoxycarbonyl, (C₁-C₆)alkylenyloxycarbonyl, (C₁-C₄)alkoxy-(C₁-C₄)alkyl, C(OH)R¹⁸R¹⁹, (C₁-C₄)alkylcarbonylamido, (C₃-C₇)cycloalkylcarbonylamido, phenylcarbonylamido, phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, (C₁-C₄)alkylsulfenyl, (C₁-C₄)alkylsulfonyl, (C₃-C₇)cycloalkyl, (C₁-C₄)alkyl optionally substituted with up to three fluoro or (C₁-C₄)alkoxy optionally substituted with up to five fluoro; said phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, in the definition of R¹⁴, R¹⁵, R¹⁶ and R¹⁷ are optionally substituted with up to three substituents independently selected from hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said imidazolyl, oxazolyl, isoxazolyl, thiazolyl and pyrazolyl in the definition of R¹⁴, R¹⁵, R¹⁶ and R¹⁷ are optionally substituted with up to two substituents independently selected from hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to three fluoro; and R¹⁸ and R¹⁹ are each independently hydrogen or (C₁-C₄)alkyl;

X and Y together are CH₂—CH(OH)—Ar or CH₂—C(O)—Ar, or

X is a covalent bond, NR²⁰ or CHR²¹, wherein, R²⁰ is (C₁-C₃)alkyl or a phenyl that is optionally substituted with one or more substituents selected from OH, F, Cl, Br, I, CN, CF₃, (C₁-C₆)alkyl, O—(C₁-C₆)alkyl, S(O)ₙ—(C₁-C₆)alkyl and SO₂—NR²²R²³, and R²¹ is hydrogen or methyl, and

Y is a phenyl or naphthyl ring optionally substituted with one or more substituents selected from Ar, OH, F, Cl, Br, I, CN, CF₃, (C₁-C₆)alkyl, O—(C₁-C₆)alkyl, S(O)ₙ—(C₁-C₆)alkyl and SO₂—NR²²R²³;

Ar is a phenyl or naphthyl ring optionally substituted with one or more substituents selected from F, Cl, Br, I, CN, CF₃, (C₁-C₆)alkyl, O—(C₁-C₆)alkyl, S(O)ₙ—(C₁-C₆)alkyl and SO₂—NR²²R²³;

n is independently for each occurrence 0, 1 or 2;

R²² is independently for each occurrence H, (C₁-C₆)alkyl, phenyl or naphthyl; and

R²³ is independently for each occurrence (C₁-C₆)alkyl, phenyl or naphthyl, provided that when R³ is NR⁶R⁷, then A is SO₂; a second dosage form comprising a second compound that is a cyclooxygenase-2 inhibitor, a prodrug of said second compound or a pharmaceutically acceptable salt of said second compound or said prodrug; and a container.

An additional aspect of this invention is therapeutic methods comprising administering to a mammal in need of treatment or prevention of diabetic complications a first compound selected from:

a compound of formula I

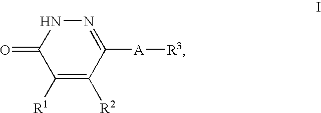

and a compound of formula II

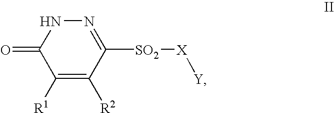

or a prodrug of said first compound, or a pharmaceutically acceptable salt of said first compound or said prodrug,

wherein:

A is S, SO or SO₂;

R¹ and R² are each independently hydrogen or methyl;

R³ is Het¹, —CHR⁴Het¹ or NR⁶R⁷;

R⁴ is hydrogen or (C₁-C₃)alkyl;

R⁶ is (C₁-C₆)alkyl, aryl or Het²;

R⁷ is Het³;

Het¹ is pyridyl, pyrimidyl, pyrazinyl, pyridazinyl, quinolyl, isoquinolyl, quinazolyl, quinoxalyl, phthalazinyl, cinnolinyl, naphthyridinyl, pteridinyl, pyrazinopyrazinyl, pyrazinopyridazinyl, pyrimidopyridazinyl, pyrimidopyrimidyl, pyridopyrimidyl, pyridopyrazinyl, pyridopyridazinyl, pyrrolyl, furanyl, thienyl, imidazolyl, oxazolyl, thiazolyl, pyrazolyl, isoxazolyl, isothiazolyl, triazolyl, oxadiazolyl, thiadiazolyl, tetrazolyl, indolyl, benzofuranyl, benzothienyl, benzimidazolyl, benzoxazolyl, benzothiazolyl, indazolyl, benzisoxazolyl, benzisothiazolyl, pyrrolopyridyl, furopyridyl, thienopyridyl, imidazolopyridyl, oxazolopyridyl, thiazolopyridyl, pyrazolopyridyl, isoxazolopyridyl, isothiazolopyridyl, pyrrolopyrimidyl, furopyrimidyl, thienopyrimidyl, imidazolopyrimidyl, oxazolopyrimidyl, thiazolopyrimidyl, pyrazolopyrimidyl, isoxazolopyrimidyl, isothiazolopyrimidyl, pyrrolopyrazinyl, furopyrazinyl, thienopyrazinyl, imidazolopyrazinyl, oxazolopyrazinyl, thiazolopyrazinyl, pyrazolopyrazinyl, isoxazolopyrazinyl, isothiazolopyrazinyl, pyrrolopyridazinyl, furopyridazinyl, thienopyridazinyl, imidazolopyridazinyl, oxazolopyridazinyl, thiazolopyridazinyl, pyrazolopyridazinyl, isoxazolopyridazinyl or isothiazolopyridazinyl; Het¹ is independently optionally substituted with up to a total of four substituents independently selected from R⁸, R⁹, R¹⁰ and R¹¹; wherein R⁸, R⁹, R¹⁰ and R¹¹ are each taken separately and are each independently halo, formyl, (C₁-C₆)alkoxycarbonyl, (C₁-C₆)alkylenyloxycarbonyl, (C₁-C₄)alkoxy-(C₁-C₄)alkyl, C(OH)R¹²R¹³, (C₁-C₄)alkylcarbonylamido, (C₃-C₇)cycloalkylcarbonylamido, phenylcarbonylamido, phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, (C₁-C₄)alkylsulfenyl, (C₁-C₄)alkylsulfonyl, (C₃-C₇)cycloalkyl, (C₁-C₄)alkyl optionally substituted with up to three fluoro or (C₁-C₄)alkoxy optionally substituted with up to five fluoro; said phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, in the definition of R⁸, R⁹, R¹⁰ and R¹¹ are optionally substituted with up to three substituents independently selected from hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said imidazolyl, oxazolyl, isoxazolyl, thiazolyl and pyrazolyl in the definition of R⁸, R⁹, R¹⁰ and R¹¹ are optionally substituted with up to two substituents independently selected from hydroxy, halo, C₁-C₄)alkyl, hydroxy-(C₁-C₄)alkyl, (C₁-C₄)alkoxy-(C₁-C₄)alkyl, C₁-C₄)alkyl-phenyl optionally substituted in the phenyl portion with one Cl, Br, OMe, Me or SO₂-phenyl wherein said SO₂-phenyl is optionally substituted in the phenyl portion with one Cl, Br, OMe, Me, (C₁-C₄)alkyl optionally substituted with up to five fluoro, or (C₁-C₄)alkoxy optionally substituted with up to three fluoro; R¹² and R¹³ are each independently hydrogen or (C₁-C₄)alkyl;

Het² and Het³ are each independently imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy; Het² and Het³ are each independently optionally substituted with up to a total of four substituents independently selected from R¹⁴, R¹⁵, R¹⁶ and R¹⁷, wherein R¹⁴, R¹⁵, R¹⁶ and R¹⁷ are each taken separately and are each independently halo, formyl, (C₁-C₆)alkoxycarbonyl, (C₁-C₆)alkylenyloxycarbonyl, (C₁-C₄)alkoxy-(C₁-C₄)alkyl, C(OH)R¹⁸R¹⁹, (C₁-C₄)alkylcarbonylamido, (C₃-C₇)cycloalkylcarbonylamido, phenylcarbonylamido, phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, (C₁-C₄)alkylsulfenyl, (C₁-C₄)alkylsulfonyl, (C₃-C₇)cycloalkyl, (C₁-C₄)alkyl optionally substituted with up to three fluoro or (C₁-C₄)alkoxy optionally substituted with up to five fluoro; said phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, in the definition of R¹⁴, R¹⁵, R¹⁶ and R¹⁷ are optionally substituted with up to three substituents independently selected from hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said imidazolyl, oxazolyl, isoxazolyl, thiazolyl and pyrazolyl in the definition of R¹⁴, R¹⁵, R¹⁶ and R¹⁷ are optionally substituted with up to two substituents independently selected from hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to three fluoro; and R¹⁸ and R¹⁹ are each independently hydrogen or (C₁-C₄)alkyl;

X and Y together are CH₂—CH(OH)—Ar or CH₂—C(O)—Ar, or

X is a covalent bond, NR²⁰ or CHR²¹, wherein, R²⁰ is (C₁-C₃)alkyl or a phenyl that is optionally substituted with one or more substituents selected from OH, F, Cl, Br, I, CN, CF₃, (C₁-C₆)alkyl, O—(C₁-C₆)alkyl, S(O)ₙ—(C₁-C₆)alkyl and SO₂—NR²²R²³, and R²¹ is hydrogen or methyl, and

Y is a phenyl or naphthyl ring optionally substituted with one or more substituents selected from Ar, OH, F, Cl, Br, I, CN, CF₃, (C₁-C₆)alkyl, O—(C₁-C₆)alkyl, S(O)ₙ—(C₁-C₆)alkyl and SO₂—NR²²R²³;

Ar is a phenyl or naphthyl ring optionally substituted with one or more substituents selected from F, Cl, Br, I, CN, CF₃, (C₁-C₆)alkyl, O—(C₁-C₆)alkyl, S(O)ₙ—(C₁-C₆)alkyl and SO₂—NR²²R²³;

n is independently for each occurrence 0, 1 or 2;

R²² is independently for each occurrence H, (C₁-C₆)alkyl, phenyl or naphthyl; and

R²³ is independently for each occurrence (C₁-C₆)alkyl, phenyl or naphthyl,

provided that when R³ is NR⁶R⁷, then A is SO₂, and a second compound that is a cyclooxygenase-2 inhibitor, a prodrug of said second compound or a pharmaceutically acceptable salt of said second compound or said prodrug.

A still further aspect of this invention is therapeutic methods comprising administering to a mammal in need of treatment or prevention of cardiac tissue ischemia a first compound selected from:

a compound of formula I

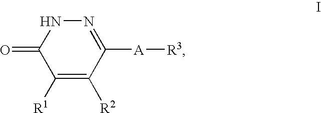

and a compound of formula II

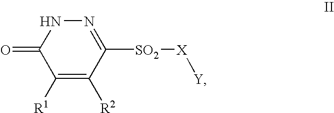

or a prodrug of said first compound, or a pharmaceutically acceptable salt of said first compound or said prodrug,

wherein:

A is S, SO or SO₂;

R¹ and R² are each independently hydrogen or methyl;

R³ is Het¹, —CHR⁴Het¹ or NR⁶R⁷;

R⁴ is hydrogen or (C₁-C₃)alkyl;

R⁶ is (C₁-C₆)alkyl, aryl or Het²;

R⁷ is Het³;

Het¹ is pyridyl, pyrimidyl, pyrazinyl, pyridazinyl, quinolyl, isoquinolyl, quinazolyl, quinoxalyl, phthalazinyl, cinnolinyl, naphthyridinyl, pteridinyl, pyrazinopyrazinyl, pyrazinopyridazinyl, pyrimidopyridazinyl, pyrimidopyrimidyl, pyridopyrimidyl, pyridopyrazinyl, pyridopyridazinyl, pyrrolyl, furanyl, thienyl, imidazolyl, oxazolyl, thiazolyl, pyrazolyl, isoxazolyl, isothiazolyl, triazolyl, oxadiazolyl, thiadiazolyl, tetrazolyl, indolyl, benzofuranyl, benzothienyl, benzimidazolyl, benzoxazolyl, benzothiazolyl, indazolyl, benzisoxazolyl, benzisothiazolyl, pyrrolopyridyl, furopyridyl, thienopyridyl, imidazolopyridyl, oxazolopyridyl, thiazolopyridyl, pyrazolopyridyl, isoxazolopyridyl, isothiazolopyridyl, pyrrolopyrimidyl, furopyrimidyl, thienopyrimidyl, imidazolopyrimidyl, oxazolopyrimidyl, thiazolopyrimidyl, pyrazolopyrimidyl, isoxazolopyrimidyl, isothiazolopyrimidyl, pyrrolopyrazinyl, furopyrazinyl, thienopyrazinyl, imidazolopyrazinyl, oxazolopyrazinyl, thiazolopyrazinyl, pyrazolopyrazinyl, isoxazolopyrazinyl, isothiazolopyrazinyl, pyrrolopyridazinyl, furopyridazinyl, thienopyridazinyl, imidazolopyridazinyl, oxazolopyridazinyl, thiazolopyridazinyl, pyrazolopyridazinyl, isoxazolopyridazinyl or isothiazolopyridazinyl; Het¹ is independently optionally substituted with up to a total of four substituents independently selected from R⁸, R⁹, R¹⁰ and R¹¹; wherein R⁸, R⁹, R¹⁰ and R¹¹ are each taken separately and are each independently halo, formyl, (C₁-C₆)alkoxycarbonyl, (C₁-C₆)alkylenyloxycarbonyl, (C₁-C₄)alkoxy-(C₁-C₄)alkyl, C(OH)R¹²R¹³, (C₁-C₄)alkylcarbonylamido, (C₃-C₇)cycloalkylcarbonylamido, phenylcarbonylamido, phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, (C₁-C₄)alkylsulfenyl, (C₁-C₄)alkylsulfonyl, (C₃-C₇)cycloalkyl, (C₁-C₄)alkyl optionally substituted with up to three fluoro or (C₁-C₄)alkoxy optionally substituted with up to five fluoro; said phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, in the definition of R⁸, R⁹, R¹⁰ and R¹¹ are optionally substituted with up to three substituents independently selected from hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said imidazolyl, oxazolyl, isoxazolyl, thiazolyl and pyrazolyl in the definition of R⁸, R⁹, R¹⁰ and R¹¹ are optionally substituted with up to two substituents independently selected from hydroxy, halo, C₁-C₄)alkyl, hydroxy-(C₁-C₄)alkyl, (C₁-C₄)alkoxy-(C₁-C₄)alkyl, C₁-C₄)alkyl-phenyl optionally substituted in the phenyl portion with one Cl, Br, OMe, Me or SO₂-phenyl wherein said SO₂-phenyl is optionally substituted in the phenyl portion with one Cl, Br, OMe, Me, (C₁-C₄)alkyl optionally substituted with up to five fluoro, or (C₁-C₄)alkoxy optionally substituted with up to three fluoro;

R¹² and R¹³ are each independently hydrogen or (C₁-C₄)alkyl;

Het² and Het³ are each independently imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy; Het² and Het³ are each independently optionally substituted with up to a total of four substituents independently selected from R¹⁴, R¹⁵, R¹⁶ and R¹⁷, wherein R¹⁴, R¹⁵, R¹⁶ and R¹⁷ are each taken separately and are each independently halo, formyl, (C₁-C₆)alkoxycarbonyl, (C₁-C₆)alkylenyloxycarbonyl, (C₁-C₄)alkoxy-(C₁-C₄)alkyl, C(OH)R¹⁸R¹⁹, (C₁-C₄)alkylcarbonylamido, (C₃-C₇)cycloalkylcarbonylamido, phenylcarbonylamido, phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, (C₁-C₄)alkylsulfenyl, (C₁-C₄)alkylsulfonyl, (C₃-C₇)cycloalkyl, (C₁-C₄)alkyl optionally substituted with up to three fluoro or (C₁-C₄)alkoxy optionally substituted with up to five fluoro; said phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, in the definition of R¹⁴, R¹⁵, R¹⁶ and R¹⁷ are optionally substituted with up to three substituents independently selected from hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said imidazolyl, oxazolyl, isoxazolyl, thiazolyl and pyrazolyl in the definition of R¹⁴, R¹⁵, R¹⁶ and R¹⁷ are optionally substituted with up to two substituents independently selected from hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to three fluoro; and R¹⁸ and R¹⁹ are each independently hydrogen or (C₁-C₄)alkyl;

X and Y together are CH₂—CH(OH)—Ar or CH₂—C(O)—Ar, or

X is a covalent bond, NR²⁰ or CHR²¹, wherein, R²⁰ is (C₁-C₃)alkyl or a phenyl that is optionally substituted with one or more substituents selected from OH, F, Cl, Br, I, CN, CF₃, (C₁-C₆)alkyl, O—(C₁-C₆)alkyl, S(O)ₙ—(C₁-C₆)alkyl and SO₂—NR²²R²³, and R²¹ is hydrogen or methyl, and

Y is a phenyl or naphthyl ring optionally substituted with one or more substituents selected from Ar, OH, F, Cl, Br, I, CN, CF₃, (C₁-C₆)alkyl, O—(C₁-C₆)alkyl, S(O)ₙ—(C₁-C₆)alkyl and SO₂—NR²²R²³;

Ar is a phenyl or naphthyl ring optionally substituted with one or more substituents selected from F, Cl, Br, I, CN, CF₃, (C₁-C₆)alkyl, O—(C₁-C₆)alkyl, S(O)ₙ—(C₁-C₆)alkyl and SO₂—NR²²R²³;

n is independently for each occurrence 0, 1 or 2;

R²² is independently for each occurrence H, (C₁-C₆)alkyl, phenyl or naphthyl; and

R²³ is independently for each occurrence (C₁-C₆)alkyl, phenyl or naphthyl,

provided that when R³ is NR⁶R⁷, then A is SO₂, and a second compound that is a cyclooxygenase-2 inhibitor, a prodrug of said second compound or a pharmaceutically acceptable salt of said second compound or said prodrug.

In a preferred embodiment of the composition, kit and method aspects of this invention said first compound is a compound of formula I, wherein A is SO₂; R¹ and R² are each hydrogen; R³ is Het¹, wherein Het¹ is 5H-furo-[3,2c]pyridin-4-one-2-yl, furano[2,3b]pyridin-2-yl, thieno[2,3b]pyridin-2-yl, indol-2-yl, indol-3-yl, benzofuran-2-yl, benzothien-2-yl, imidazo[1,2a]pyridin-3-yl, pyrrol-1-yl, imidazol-1-yl, indazol-1-yl, tetrahydroquinol-1-yl or tetrahydroindol-1-yl, wherein said Het¹ is optionally independently substituted with up to a total of two substituents each independently selected from fluoro, chloro, bromo, (C₁-C₆)alkyl, (C₁-C₆)alkoxy, trifluoromethyl, hydroxy, benzyl or phenyl; said benzyl and phenyl are each optionally independently substituted with up to three halo, (C₁-C₆)alkyl, (C₁-C₆)alkoxy, (C₁-C₆)alkylsulfonyl, (C₁-C₆)alkylsulfinyl, (C₁-C₆)alkylsulfenyl, trifluoromethyl or hydroxy, or a prodrug thereof or a pharmaceutically acceptable salt of said compound or prodrug. In a more preferred embodiment, Het¹ is indol-2-yl, benzofuran-2-yl, benzothiophen-2-yl, furano[2,3b]pyridin-2-yl, thieno[2,3b]pyridin-2-yl or imidazo[1,2a]pyridin-4-yl, wherein said Het¹ is optionally independently substituted with up to a total of two substituents independently selected from fluoro, chloro, bromo, (C₁-C₆)alkyl, (C₁-C₆)alkoxy, trifluoromethyl and phenyl; said phenyl being optionally substituted with up to two substituents independently selected from fluoro, chloro and (C₁-C₆)alkyl.

In another preferred embodiment of the composition, kit and method aspects of this invention said first compound is selected from:

6-(3-trifluoromethyl-benzenesulfonyl)-2H-pyridazin-3-one;

6-(4-bromo-2-fluoro-benzenesulfonyl)-2H-pyridazin-3-one;

6-(4-trifluoromethyl-benzenesulfonyl)-2H-pyridazin-3-one;

6-(2-bromo-benzenesulfonyl)-2H-pyridazin-3-one;

6-(3,4-dichloro-benzenesulfonyl)-2H-pyridazin-3-one;

6-(4-methoxy-benzenesulfonyl)-2H-pyridazin-3-one;

6-(3-bromo-benzenesulfonyl)-2H-pyridazin-3-one;

6-(biphenyl-4-sulfonyl)-2H-pyridazin-3-one;

6-(4′-fluoro-biphenyl-4-sulfonyl)-2H-pyridazin-3-one;

6-(4′-trifluoromethyl-biphenyl-4-sulfonyl)-2H-pyridazin-3-one;

6-(3′,5′-bis-trifluoromethyl-biphenyl-4-sulfonyl)-2H-pyridazin-3-one;

6-(biphenyl-2-sulfonyl)-2H-pyridazin-3-one;

6-(4′-trifluoromethyl-biphenyl-2-sulfonyl)-2H-pyridazin-3-one;

6-(2-hydroxy-benzenesulfonyl)-2H-pyridazin-3-one;

6-(2-chloro-benzenesulfonyl)-2H-pyridazin-3-one;

6-(3-chloro-benzenesulfonyl)-2H-pyridazin-3-one;

6-(2,3-dichloro-benzenesulfonyl)-2H-pyridazin-3-one;

6-(2,5-dichloro-benzenesulfonyl)-2H-pyridazin-3-one;

6-(4-fluoro-benzenesulfonyl)-2H-pyridazin-3-one;

6-(4-chloro-benzenesulfonyl)-2H-pyridazin-3-one;

6-(2-fluoro-benzenesulfonyl)-2H-pyridazin-3-one;

6-(2,3-difluoro-benzenesulfonyl)-2H-pyridazin-3-one;

6-(2,4-dichloro-benzenesulfonyl)-2H-pyridazin-3-one;

6-(2,4-difluoro-benzenesulfonyl)-2H-pyridazin-3-one;

6-(2,6-dichloro-benzenesulfonyl)-2H-pyridazin-3-one;

6-(2-chloro-4-fluoro-benzenesulfonyl)-2H-pyridazin-3-one;

6-(2-bromo-4-fluoro-benzenesulfonyl)-2H-pyridazin-3-one; and

6-(naphthalene-1-sulfonyl)-2H-pyridazin-3-one,

or a prodrug thereof or a pharmaceutically acceptable salt of said compound or said prodrug.

In an additional preferred embodiment of the composition, kit and method aspects of this invention said second compound is selected from celecoxib, rofecoxib and etoricoxib or a prodrug thereof or a pharmaceutically acceptable salt of said compound or said prodrug.

In a preferred embodiment of the composition aspects of this invention, the composition further comprises a vehicle, diluent or carrier.

In a preferred embodiment of the composition and kit aspects of this invention, said first compound is present in an aldose reductase inhibiting amount.

In another preferred embodiment of the composition and kit aspects of this invention, said second compound is present in a cyclooxygenase-2 inhibiting amount.

In a preferred embodiment of the therapeutic method aspects of this invention said mammal is a human.

In a preferred embodiment of the therapeutic method aspects of this invention comprising administering a first compound and a second compound, said first compound is administered in an aldose reductase inhibiting amount.

In another preferred embodiment of the therapeutic method aspects of this invention comprising administering a first compound and a second compound, said second compound is administered in a cyclooxygenase-2 inhibiting amount.

In a preferred embodiment of the of the therapeutic method aspects of this invention comprising administering to a mammal in need of treatment or prevention of cardiac tissue ischemia a compound of formula II, said compound of formula II is administered in an aldose reductase inhibiting amount.

The term “alkylene” means saturated hydrocarbon (straight chain or branched) wherein a hydrogen atom is removed from each of the terminal carbons. Exemplary of such groups (assuming the designated length encompasses the particular example) are methylene, ethylene, propylene, butylene, pentylene, hexylene, heptylene.

The term “aryl” means a carbon-containing aromatic ring. Examples of aryl groups include phenyl and naphthyl.

The term “compounds of this invention”, as used herein means compounds of formula I, compounds of formula II, cyclooxygenase-2 inhibitors, and includes prodrugs of such compounds and pharmaceutically acceptable salts of such compounds and prodrugs. The terms “compound(s) of formula I”, “compound(s) of formula II” and “cyclooxygenase-2 inhibitor(s)” are meant to include prodrugs of such compounds and pharmaceutically acceptable salts of such compounds and such prodrugs.

The term “(C₁-Cₜ)alkyl” as used herein, wherein the subscript “t” denotes an integer greater than 1, denotes a saturated monovalent straight or branched aliphatic hydrocarbon radical having one to t carbon atoms.

The expression “pharmaceutically acceptable salt” as used herein in relation to compounds of this invention includes pharmaceutically acceptable cationic salts. The expression “pharmaceutically-acceptable cationic salts” is intended to define but is not limited to such salts as the alkali metal salts, (e.g., sodium and potassium), alkaline earth metal salts (e.g., calcium and magnesium), aluminum salts, ammonium salts, and salts with organic amines such as benzathine (N,N′-dibenzylethylenediamine), choline, ethanolamine, diethanolamine, triethanolamine, ethylenediamine, meglumine (N-methylglucamine), benethamine (N-benzylphenethylamine), ethanolamine, diethylamine, piperazine, triethanolamine (2-amino-2-hydroxymethyl-1,3-propanediol) and procaine.

Pharmaceutically acceptable salts of the compounds of formula I and formula II of this invention may be readily prepared by reacting the free acid form of said compounds with an appropriate base, usually one equivalent, in a co-solvent. Preferred co-solvents include diethylether, diglyme and acetone. Preferred bases include sodium hydroxide, sodium methoxide, sodium ethoxide, sodium hydride, potassium methoxide, magnesium hydroxide, calcium hydroxide, benzathine, choline, ethanolamine, diethanolamine, piperazine and triethanolamine. The salt is isolated by concentration to dryness or by addition of a non-solvent. In many cases, salts may be prepared by mixing a solution of the acid with a solution of a different salt of the cation (e.g., sodium or potassium ethylhexanoate, magnesium oleate) and employing a co-solvent, as described above, from which the desired cationic salt precipitates, or can be otherwise isolated by concentration.

The term “prodrug” denotes a compound that is converted in vivo into a compound having a particular pharmaceutically activity. Such compounds include N-alkyl derivatives and O-alkyl derivatives. For example such compounds include N-alkyl derivatives of the compounds of formula I and formula II compounds and O-alkyl derivatives of formula I and formula II tautomeric compounds.

The terms “sulfenyl”, “sulfinyl” and “sulfonyl” mean S, SO, SO₂, respectively.

The terms “DMF”, “DMSO” and “THF” mean N,N-dimethylformamide, dimethyl sulfoxide and tetrahydrofuran, respectively.

It is intended that all possible points of attachment are meant if a carbocyclic or heterocyclic moiety may be bonded or otherwise attached to a designated substrate through differing ring atoms without denoting a specific point of attachment, whether through a carbon atom or, for example, a trivalent nitrogen atom. For example, the term “pyridyl” means 2-, 3-, or 4-pyridyl, the term “thienyl” means 2-, or 3-thienyl, and so forth.

Those skilled in the art will recognize that the compounds of this invention can exist in several tautomeric forms. All such tautomeric forms are considered as part of this invention. For example, all of the tautomeric forms of the carbonyl moiety of the compounds of formula II are included in this invention. Also, for example all enol-keto forms of compounds of formula I and the compounds of formula II are included in this invention.

Those skilled in the art will also recognize that the compounds of this invention can exist in several diastereoisomeric and enantiomeric forms. All diastereoisomeric and enantiomeric forms, and racemic mixtures thereof, are included in this invention.

Those skilled in the art will further recognize that the compounds of formula I and formula II can exist in crystalline form as hydrates wherein molecules of water are incorporated within the crystal structure thereof and as solvates wherein molecules of a solvent are incorporated therein. All such hydrate and solvate forms are considered part of this invention.

This invention also includes isotopically-labeled compounds, which are identical to those described by formula I and formula II, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, sulfur, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, may afford certain therapeutic advantages resulting from greater metabolic stability, for example increased in vivo half-life or reduced dosage requirements and, hence, may be preferred in some circumstances. Isotopically labeled compounds of formula I and formula II of this invention and prodrugs thereof can generally be prepared by carrying out the procedures disclosed in the schemes and/or in the Examples below, by substituting a readily available isotopically labeled reagent for a non-isotopically labeled reagent.

### DETAILED DESCRIPTION OF THE INVENTION

In general, the compounds of formula I and formula II of this invention may be prepared by methods that include processes analogous to those known in the chemical arts, particularly in light of the description contained herein. Certain processes for the manufacture of the compounds of formula I and formula II of this invention are illustrated by the following reaction schemes. Other processes are described in the experimental section.

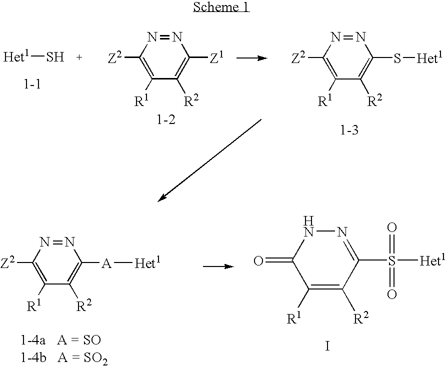

According to Scheme 1, compounds of Formula I, wherein R¹ and R² are as defined above and R³ is Het¹, can be prepared from the corresponding pyridazine of formula 1-2 and a heterocyclic thiol of formula 1-1. A thiol 1-1, in which R³ of the compounds of Formula I is Het¹, is reacted with a base such as an alkali metal (C₁-C₆)alkoxide in a (C₁-C₆) alkanol, to obtain the alkali metal salt of said thiol. Preferred alkali metal (C₁-C₆)alkoxides include, but are not limited to, sodium methoxide, sodium ethoxide and potassium t-butoxide. After evaporating the excess solvent, the resulting alkali metal salt of said thiol is refluxed with a compound of formula 1-2 wherein Z¹ and Z² are each independently selected from chloro, (C₁-C₆)alkoxy, phenyloxy or benzyloxy, said benzyloxy or phenyloxy being optionally substituted with one or two chloro or methyl groups in an aromatic hydrocarbon solvent or solvent system, for example, toluene, benzene or xylene. The reaction is allowed to stir overnight to obtain a compound of formula 1-3. The reaction is usually conducted at ambient pressure and at the refluxing temperature of the solvent used. Compounds of formula 1-3 can also be prepared by reacting compounds 1-2, wherein R¹, R², Z¹ and Z² are as defined above with a compound of formula 1-1 in a reaction inert solvent such as a polar non-aqueous solvent containing an alkali or alkali earth metal hydride or an alkali or alkali earth (C₁-C₄)alkoxide. Preferred such solvents include, but are not limited to, acetonitrile and ether solvents such as diglyme, tetrahydrofuran (THF) and dimethylformamide (DMF). Preferred such alkali or alkali earth metal hydrides include, but are not limited to, sodium hydride. Preferred alkali or alkali earth metal (C₁-C₄)alkoxides include, but are not limited to, potassium t-butoxide. The preferred metal hydride is sodium hydride. A particularly preferred solvent is DMF. Compounds of formula 1-3 can also be prepared by reacting a compound of formula 1-1 with a compound of formula 1-2, wherein the variables are as defined above, in a reaction inert solvent such as DMF, THF, diglyme or dioxane containing sodium carbonate, potassium carbonate, sodium bicarbonate or potassium bicarbonate. This reaction is usually conducted at ambient pressure and at temperatures between about 60° C. and about 120° C. A compound of formula 1-3 can be oxidized to afford a sulfoxide or a sulfonyl compound of formula 14a and/or 14b, respectively. A preferred procedure is oxidation of a compound of formula 1-3 with 30% hydrogen peroxide in the presence or absence of an organic acid such as formic acid or acetic acid. Another preferred oxidation procedure involves the use of peracid in the corresponding organic acid as solvent. Yet another preferred procedure is oxidation of a compound of formula 1-3 with a peracid, for example meta-chloroperbenzoic acid (MCPBA), in a halocarbon solvent, for example, methylene chloride, chloroform or ethylene chloride. In any case, the reaction is conducted at ambient pressure and at temperatures between about 20° C. and about −40° C. with careful reaction monitoring to avoid formation of N-oxides by over-oxidation at the nitrogen atom. The oxidation reaction is usually complete within three to six hours and proceeds through sulfoxide 1-4a, but occasionally may be complete prior to the passage of three hours, as determined by a person skilled in the art. If the reaction is conducted at between about 20° C. and about 30° C., and is stopped at between one to three hours, sulfoxide 1-4a can be isolated using separation procedures well known to a person skilled in the art. The resulting sulfone of formula 14b can then be hydrolyzed with a mineral acid such as, but not limited to, concentrated hydrochloric acid with no solvent or in a reaction inert solvent such as an ether solvent, for example, dioxane, tetrahydrofuran or diethyl ether, to obtain a compound of Formula I. The hydrolysis reaction is generally conducted at ambient pressure and at the refluxing temperature of the solvent used.

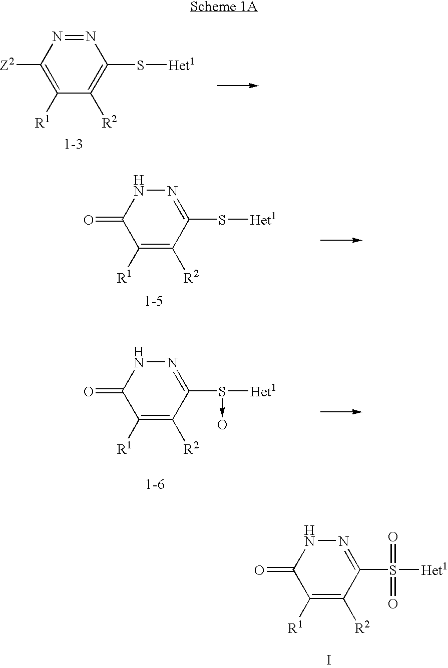

According to Scheme IA, compounds of Formula I can also be prepared by reversing the order of the last two steps of Scheme I, i.e., by formation of the oxo compound of Formula I prior to oxidation of the sulfide of formula 1-5 to the sulfone of Formula I via the sulfoxide of Formula 1-6. Thus, a compound of formula 1-3 is hydrolyzed in the manner described above to afford a pyridazinone compound of formula 1-5, which is then oxidized in the manner described above to afford a compound of Formula I. Compounds of formula 1-6 can also be prepared by hydrolyzing compounds of formula 1-4a as described for Scheme 1.

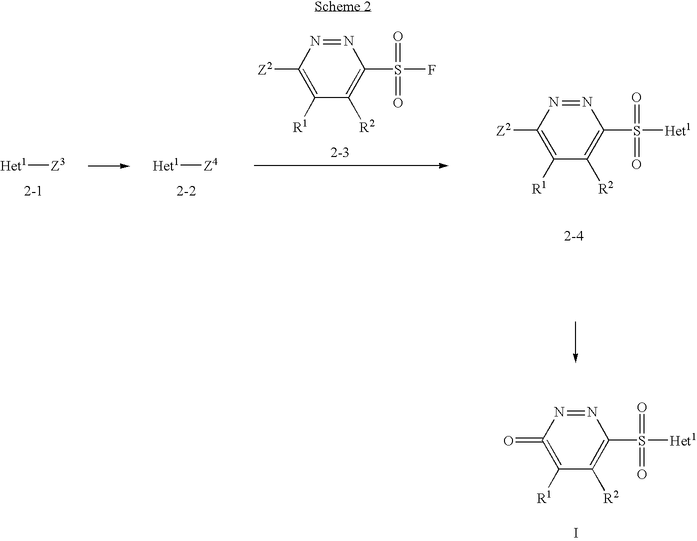

According to Scheme 2, compounds of Formula I can be prepared by reacting compounds of the formula Het¹-Z³ where Z³ is bromide, iodide or an acidic hydrogen with a suitable organometallic base to form compounds of the formula Het¹-Z⁴ wherein Z⁴ is the cation corresponding to the organometallic base. Het¹-Z⁴ may in turn may be reacted with a fluorosulfonyl pyridazine compound of the formula 2-3 to form a sulfonyl pyridazine of the formula 24 which may be hydrolyzed to form a compound of Formula I. In the case where Z³ is an acidic hydrogen, the hydrogen will be acidic enough such that said hydrogen is removable by reaction with a base such as, but not limited to, (C₁-C₆)alkyllithium, lithium diisopropylamide (LDA) or phenyl lithium. Thus, a compound of formula 2-1 in which Z³ is bromide, iodide or a hydrogen of sufficient acidity, is reacted with a base such as, but not limited to, (C₁-C₆)alkyllithium, lithium diisopropylamide (LDA) or phenyl lithium to prepare a compound of formula 2-2, wherein Z⁴ is lithium. A hydrogen of sufficient acidity is a hydrogen that can be removed from Het¹-Z³ by the bases mentioned in the preceding sentence. The reaction is conducted in a reaction inert solvent such as an ether or a hydrocarbon solvent or a mixture of such solvents. Preferred solvents include, but are not limited to, diethyl ether, tetrahydrofuran, diglyme, benzene and toluene or mixtures thereof. The reaction is conducted at temperatures from about −78° C. to about 0° C. and at ambient pressure. A compound of formula 2-2 is reacted with a compound of formula 2-3 wherein Z² is chloro, (C₁-C₆)alkoxy, phenyloxy or benzyloxy, said phenyloxy or benzyloxy being optionally substituted with one or two chloro or methyl groups to form compounds of formula 2-4 wherein Z² is as defined above. The reaction is conducted in a reaction inert solvent such as an ether or a hydrocarbon solvent or a mixture of such solvents. Preferred solvents include, but are not limited to, diethyl ether, tetrahydrofuran, diglyme, benzene and toluene or mixtures thereof. The reaction is conducted at temperatures ranging from about −78° C. to about 0° C. and at ambient pressure. Compounds 2-4 are hydrolyzed to form compounds of Formula I as described above.

Also according to Scheme 2, compounds of formula 24 may be prepared by reacting a compound of formula 2-2 wherein Z⁴ is MgBr or Mgl using standard Grignard reaction conditions, e.g., by reacting a compound of formula 2-1 wherein Z³ is bromide or iodide with magnesium to form the compound of formula 2-2 which is reacted, preferably in situ, with a compound of formula 2-3 wherein Z² is as defined above. The reaction is generally conducted in a reaction inert solvent such as an ether or a hydrocarbon solvent or a mixture of such solvents. Preferred solvents include, but are not limited to, diethyl ether, tetrahydrofuran, diglyme, benzene and toluene or mixtures thereof. The reaction temperature ranges from about −10° C. to about 40° C. Formation of the Grignard reagent of formula 2-2 may be readily accomplished according to methods well known to those skilled in the art.

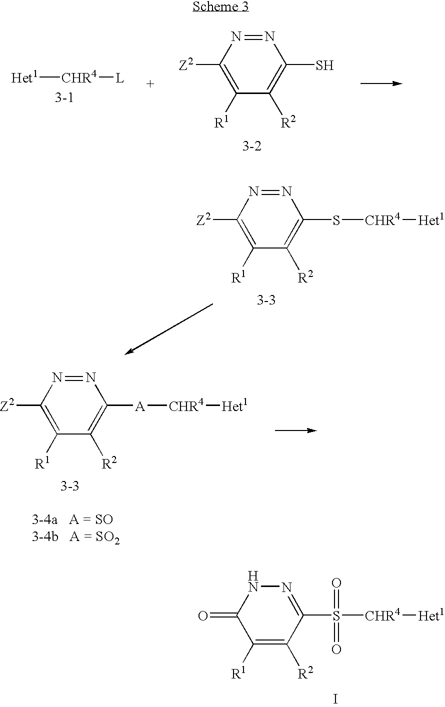

According to Scheme 3, compounds of Formula I wherein R¹, R², Z² and Het¹ are defined as described above and R³ is CHR⁴-Het¹ may be prepared by reacting a compound of the formula 3-1 with a compound of the formula 3-2 followed by further modification. Thus, a compound of the formula 3-1 wherein L is a leaving group such as chloro, bromo, iodo, methanesulfonyloxy, phenylsulfonyloxy wherein said phenyl of said phenylsulfonyloxy may be optionally substituted by one nitro, chloro, bromo or methyl is reacted with a compound of the formula 3-2, wherein Z² is as described above, to form a compound of the formula 3-3. The reaction is conducted in a reaction inert solvent such as methylene chloride, chloroform, diethyl ether, tetrahydrofuran, dioxane, acetonitrile or dimethylformamide at a temperature ranging from about room temperature to about 90° C. The reaction is conducted at ambient pressure. A compound of the formula 3-3 is then oxidized to form a sulfoxide or sulfonyl compound of the formula 3-4a and/or 34b, respectively, by reacting said compound of formula 3-3 with an oxidizing agent such as metachloroperbenzoic acid (MCPBA) in a reaction inert solvent or hydrogen peroxide in acetic acid. The sulfoxide of formula 3-4a may be isolated by halting the oxidation reaction as described in Scheme 1 above. When MCPBA is used, preferred reaction inert solvents include such solvents as methylene chloride and chloroform. The reaction is ordinarily performed at room temperature. When hydrogen peroxide is used as the oxidizing agent, the reaction is carried out as described above. Compounds of formula 3-4b thus prepared may be hydrolyzed to form compounds of Formula I according to conditions described in Scheme 1 above.

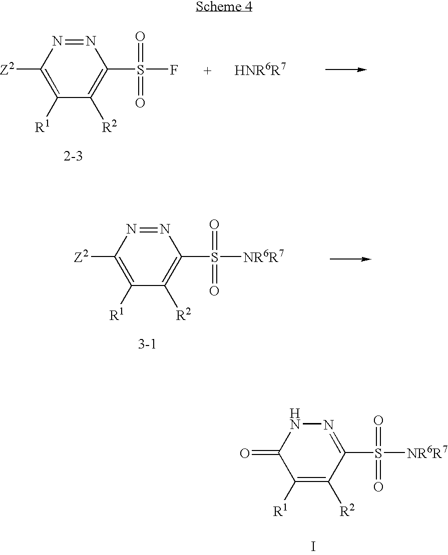

According to Scheme 4, compounds of Formula I wherein R¹, R² and Z are defined as set forth above and R³ is —NR⁶R⁷ may be prepared from compounds of formula 2-3. Thus, a compound of formula 2-3 is reacted with an amine of the formula HNR⁶R⁷, wherein R⁶ and R⁷ are defined as set forth above, in the presence of excess HNR⁶R⁷ or a tertiary amine such as, but not limited to, triethyl amine or diisopropyl ethyl amine in a reaction inert solvent to form a compound of the formula 3-1. Preferred reaction inert solvents for this reaction include, but are not limited to, methylene chloride, chloroform, diethyl ether, tetrahydrofuran and dioxane. The reaction is preferably conducted at a temperature ranging from about 0° C. to about 100° C. Compounds of formula 3-1 thus prepared may be hydrolyzed to form compounds of Formula I as described above.

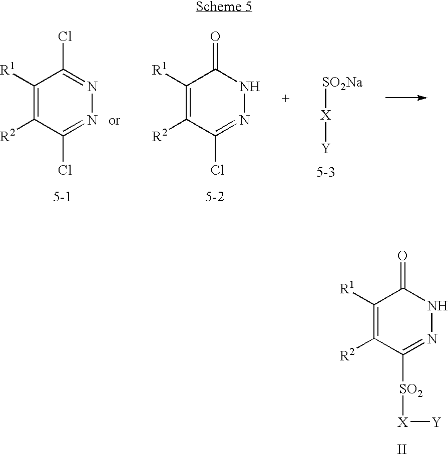

According to Scheme 5, compounds of formula II may be prepared by reacting dichloro pyridazine compounds of formula 5-1 or chloropyridazinone compounds of formula 5-2 with an alkali or alkali metal salt of Y—X—SO₂H, for example, Y—X—SO₂Na of formula 5-3, wherein R¹, R², X and Y are as defined herein. The reaction may be carried out in water or a mixture of water and water-miscible solvents such as dioxane or tetrahydrofuran (THF). The reaction is usually conducted at ambient pressure and at temperatures between about 80° C. and the boiling point of the solvent used.

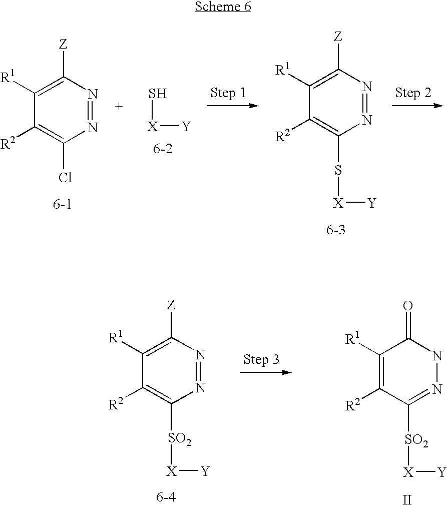

Compounds of formula II may also be prepared in accordance with the steps of Scheme 6. In step 1 of Scheme 6, a compound of formula 6-1, wherein R¹, R², X and Y are as defined herein and Z is Cl, O—(C₁-C₆)alkyl, O—Ph, O—CH₂—Ph, wherein Ph is phenyl optionally mono- or di-substituted with chlorine, bromine, or methyl, is reacted with a thiol compound of formula 6-2 to form the formula 6-3 sulfenyl compound.

In one method of step 1 of Scheme 6, a formula 6-1 compound is reacted with the alkali metal salt of a formula 6-2 thiol. The alkali metal salt is prepared by reacting the formula 6-2 thiol with an alkali metal (C₁-C₆)alkoxide in (C₁-C₆)alkyl-OH. It is preferable that the (C₁-C₆)alkoxide and the (C₁-C₆)alkyl-OH correspond to Z of the formula 6-1 compound. For example, when Z is OMe the preferred alkoxide is an alkali metal methoxide, preferably sodium methoxide, and the preferred (C₁-C₆)alkyl-OH is methanol. Potassium t-butoxide may be used in any combination of alkanol and Z. Preferred metal oxides are sodium methoxide and sodium ethoxide. Excess alcohol from the reaction forming the alkali metal salt of the formula 6-2 thiol compound is evaporated away and the resulting alkali metal salt is refluxed overnight in an aromatic hydrocarbon solvent, preferably toluene, together with the formula 6-1 compound to form the formula 6-3 compound.

In another method of step 1 of Scheme 6, compounds of formula 6-3 may be prepared by reacting compounds of formula 6-1 with compounds of formula 6-2 in N,N-dimethylformamide (DMF) containing sodium or potassium carbonate. The reaction is preferably conducted at ambient pressure and at a temperature of between about 60° C. and about 120° C.

In a further method of step 1 of Scheme 6, compounds of formula 6-1, wherein Z is O—(C₁-C₆)alkyl, are reacted with compounds of formula 6-2 either in a polar non-aqueous solvent (e.g., acetonitrile) or in an ether solvent (e.g., diglyme, tetrahydrofuran or DMF) containing alkali or alkali earth metal hydrides, preferably sodium hydride, or potassium t-butoxide. A preferred solvent is DMF.

Compounds of formula 6-1 of Scheme 6, wherein Z is O—(C₁-C₆)alkyl, O—Ph, O—CH₂—Ph, wherein Ph is phenyl optionally mono- or di-substituted with chlorine, bromine, or methyl, may be prepared by reacting a compound of formula 5-1

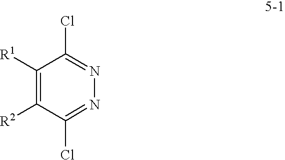

with the sodium salts of HO—(C₁-C₆)alkyl, HO—Ph or HO—CH₂—Ph. The sodium salts may be prepared by reacting HO—(C₁-C₆)alkyl, HO—Ph or HO—CH₂—Ph, as applicable, with sodium metal at a temperature of about 0° C. to about 50° C. The oxide may also be prepared by reacting HO—(C₁-C₆)alkyl, HO—Ph or HO—CH₂—Ph with sodium hydride, optionally in the presence of a reaction-inert solvent, preferably benzene, toluene, THF or ether, at a temperature of between about 0° C. and about room temperature.

In step 2 of Scheme 6, a compound of formula 6-3 is oxidized to form the formula 64 sulfonyl compound. The formula 6-3 compounds may be oxidized with 30% hydrogen peroxide, optionally in the presence of formic acid, acetic acid or a peracid, such as m-chloroperbenzoic acid (MCPBA), in a halocarbon solvent (e.g., dichloromethane). The reaction is preferably conducted at ambient pressure and at a temperature of between about 20° C. and about 40° C., and is complete in about three to about six hours. The reaction should be monitored carefully to avoid over-oxidation of the nitrogen atoms to N-oxides. N-oxides that are formed may be converted to the reduced pyridazine compound by reacting the N-oxide with triethylphosphite, sodium sulfite or potassium sulfite, preferably at about 100° C. for about four hours.

The formula 64 compounds of step 3 of Scheme 6 are hydrolyzed with a mineral acid, e.g., concentrated hydrochloric acid, alone or in an ether solvents such as dioxane, to obtain the compound of formula II. The reaction of step 3 is preferably conducted at ambient pressure and at the refluxing temperature of the solvent used.

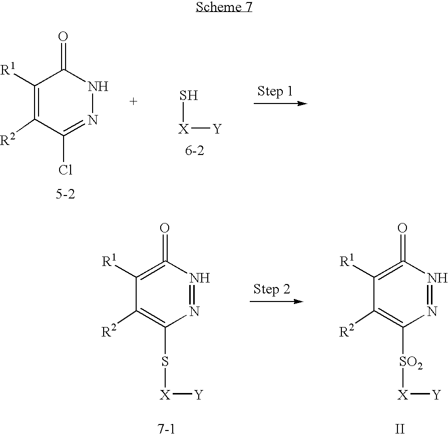

Scheme 7 provides still another method of preparing compounds of formula II. In Scheme 7, a chloropyridazinone compound of formula 5-2 is reacted with a thiol compound of formula 6-2 to form a sulfinylpyridazinone compound of formula 7-1. The reaction is preferably performed in the presence of an alkali or an alkali metal alkoxide, for example potassium tertbutoxide, in reaction-inert polar solvent such as DMF or acetonitrile at about room temperature to about 100° C. The resulting compound of formula 7-1 is oxidized with hydrogen peroxide, optionally in the presence of acetic acid or a peracid, preferably m-chloroperbenzoic acid (MCPBA), in a halocarbon solvent such as dichloromethane, to form the compound of formula II.

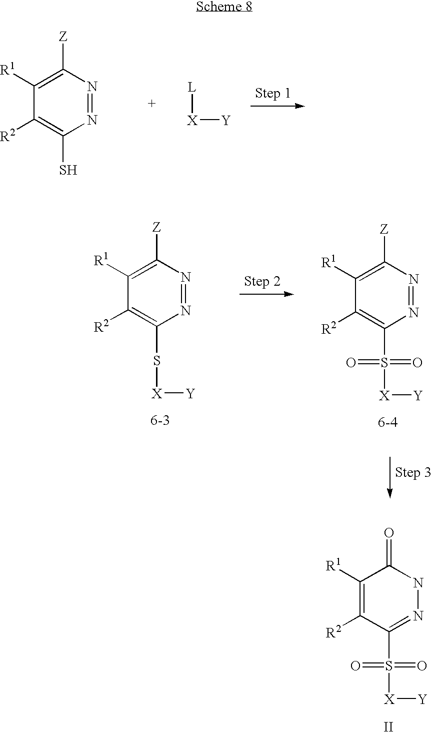

Compounds of formula II wherein X is CHR²¹, wherein R²¹ is hydrogen or methyl may be prepared according to Scheme 8. In step 1 of Scheme 8, a compound of formula 8-1, wherein Z is Cl, O—(C₁-C₆)alkyl, O—Ph¹, O—CH₂—Ph¹, wherein Ph¹ is phenyl optionally mono- or di-substituted with chlorine, bromine, or methyl, is reacted with Y—X—L, wherein L is a leaving group, preferably Cl, Br, I, OSO₂CH₃, OSO₂CF₃, or OSO₂Ph², wherein Ph² is a phenyl optionally monosubtituted with Br, Cl or OCH₃, in the presence of a base, preferably sodium carbonate, potassium carbonate or sodium hydride to form a compound of formula 6-3. When the base is sodium carbonate or potassium carbonate, the reaction solvent is preferably acetone. However, if the base is sodium hydride, DMF or acetonitrile is used as the reaction solvent. The reaction is preferably conducted at ambient pressure and at a temperature of between about room temperature and about 100° C. Steps 2 and 3 are analogous to steps 2 and 3 of Scheme 6 and are conducted in the same manner thereof.

Compounds of formula II wherein X and Y together form —CH₂C(O)Ar may be prepared according to Scheme 8 by reacting, in step 1, compounds of formula 8-1 with LCH₂C(O)Ar to form a compound of formula 6-3. The reaction is conducted in the presence of a base, preferably sodium carbonate or potassium carbonate and in a reaction-inert solvent such as dimethyl formamide. The reaction temperature is preferably from about room temperature to about 80° C. Steps 2 and step 3 of Scheme 8 are performed in a manner analogous to steps 2 and 3 of Scheme 6.

Compounds of formula II wherein X and Y together form —CH₂CH(OH)Ar may be prepared by reacting compounds of formula II wherein X and Y together form —CH₂C(O)Ar with sodium borohydride in alcoholic solvents such as methanol, ethanol or isopropanol. The reaction is preferably conducted at a temperature of about 0° C. to about 60° C. and at ambient pressure.

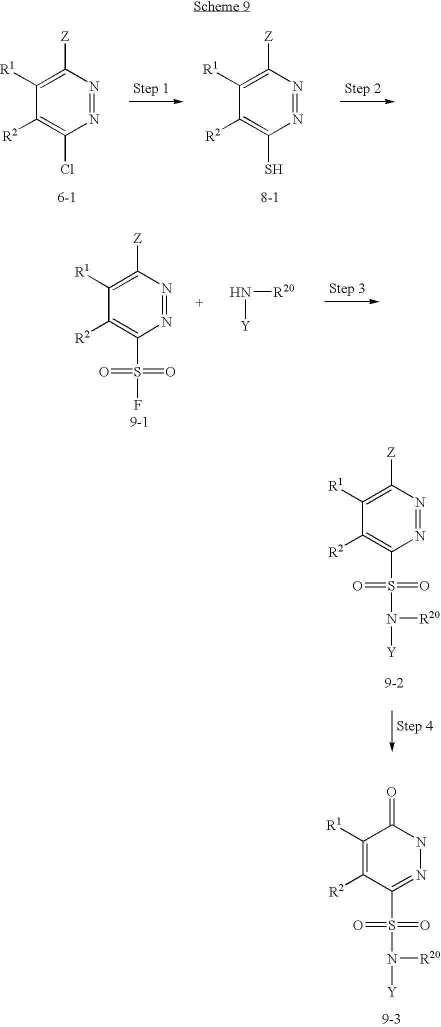

Compounds of formula II wherein X is NR²⁰ wherein R²⁰ is (C₁-C₃)alkyl (formula 9-3 compounds) may be prepared in accordance with Scheme 9. In step 1 of Scheme 9, a compound of formula 6-1, wherein Z is Cl, O—(C₁-C₆)alkyl, O—Ph, O—CH₂—Ph, wherein Ph is phenyl optionally mono- or di-substituted with chlorine, bromine, or methyl, is reacted with thiourea in a ketone solvents, preferably acetone, ethyl methyl ketone or isobutyl ketone, to obtain a compound of formula 8-1. Step 1 is conducted at ambient pressure and at the refluxing temperature of the solvent. Compounds of formula 6-1 may be prepared as described above for Scheme 6.

In step 2 of Scheme 9, a compound of formula 9-1 is prepared according to the process disclosed in J. Heterocyclic Chem., 1998, 35, 429-436. Compounds of formula 9-1 are particularly useful as intermediates in the preparation of compounds of formula II.

In Step 3 of Scheme 9, a formula 9-2 compound is prepared by reacting a compound of formula 9-1 with excess HN(R²⁰)—Y, optionally in an organic reaction inert base, preferably a trialkyl amine selected from trimethylamine, triethylamine, and dimethyl-isopropyl-amines, more preferably triethylamine. The reaction may optionally be performed in a reaction inert solvent such as an ether, halocarbon or aromatic hydrocarbon solvent, preferably selected from diethyl ether, isopropyl ether, tetrahydrofuran, diglyme, chloroform, methylene dichloride, benzene and toluene. The reaction of step 3 is preferably performed at a temperature of about room temperature to about the refluxing temperature of the solvent that is used.

In step 4 of Scheme 9, a compound of formula 9-3 may be prepared by hydrolyzing a compound of formula 9-2 with a mineral acid such as concentrated hydrochloric acid, either alone or an ether solvent (e.g., dioxane). The reaction may be conducted at about room pressure to about the refluxing temperature of the solvent used.

Compounds of formula II wherein X is a covalent bond and Y is a phenyl or napthyl ring substituted with hydroxy may be prepared by reacting compounds of formula II wherein Y is phenyl or naphthyl substituted with C₁-C₆ alkoxy with a dealkylating reagents such as AlCl₃, AlBr3, or BF₃. When AlCl₃ or AlBr₃ are the dealkylating reagent, the reaction is preferably carried out without any solvent. When the dealkylating reagent is BF₃, a halocarbon solvent is preferably used, preferably methylene chloride or ethylene chloride. The reaction is conducted at ambient pressure and at temperatures between about −60° C. to about 80° C.

Compounds of formula II wherein X is a covalent bond and Y is phenyl or naphthyl substituted with an optionally substituted phenyl or naphthyl ring may be prepared by first reacting compounds of formula 6-4 wherein X is a covalent bond, Z is O—(C₁-C₆)alkyl, Y is a phenyl or napthyl that has a bromo or iodo substitutent with an appropriately substituted phenyl or naphthyl boronic acid in the presence of a palladium catalyst such as Pd[P(Ph)₃]₄ and in the presence of either potassium carbonate or sodium carbonate. The reaction is preferably conducted in an aromatic hydrocarbon solvent, preferably toluene, or in a C₁-C₆ alcohol, preferably ethanol, at ambient pressure and at a temperature of about room temperature to the refluxing temperature of the solvent used. The product of the first step is hydrolyzed with a mineral acid, preferably hydrochloric acid, alone or an ether solvent, preferably dioxane, to obtain a compound of formula II wherein Y is phenyl or naphthyl substituted with an optionally substituted phenyl or naphthyl ring.

Cardioprotection, as indicated by a reduction in infarcted myocardium, can be induced pharmacologically using adenosine receptor agonists in isolated, retrogradely perfused rabbit hearts as an in vitro model of myocardial ischemic preconditioning (Liu et al., Cardiovasc. Res., 28:1057-1061, 1994). The in vitro test described below demonstrates that a test compound (i.e., a compound as claimed herein) can also pharmacologically induce cardioprotection, i.e., reduced myocardial infarct size, when administered to a rabbit isolated heart. The effects of the test compound are compared to ischemic preconditioning and the A1/A3 adenosine agonist, APNEA 2-(4-aminophenyl)ethyl adenosine), that has been shown to pharmacologically induce cardioprotection in the rabbit isolated heart (Liu et al., Cardiovasc. Res., 28:1057-1061,1994). The exact methodology is described below.

The protocol used for these experiments closely follows that described by Liu et al., Cardiovasc. Res., 28:1057-1061, 1994. Male New Zealand White rabbits (3-4 kg) are anesthetized with sodium pentobarbital (30 mg/kg, i.v.). After deep anesthesia is achieved (determined by the absence of an ocular blink reflex) the animal is intubated and ventilated with 100% O₂ using a positive pressure ventilator. A left thoracotomy is performed, the heart exposed, and a snare (2-0 silk) is placed loosely around a branch of the left anterior descending coronary artery, approximately ⅔ of the distance towards the apex of the heart. The heart is removed from the chest and rapidly (<30 seconds) mounted on a Langendorff apparatus. The heart is retrogradely perfused via the aorta in a non-recirculating manner with a modified Krebs solution (NaCl 118.5 mM, KCl 4.7 mM, MgSO₄ 1.2 mM, KH₂PO₄ 1.2 mM, NaHCO₃ 24.8 mM, Cacl₂ 2.5 mM, and glucose 10 mM), at a constant pressure of 80 mmHg and a temperature of 37° C. Perfusate pH is maintained at 7.4-7.5 by bubbling with 95% O₂/5% CO₂. Heart temperature is tightly controlled by using heated reservoirs for the physiological solution and water jacketing around both the perfusion tubing and the isolated heart. Heart rate and left ventricular pressures are determined via a latex balloon which is inserted in the left ventricle and connected by stainless steel tubing to a pressure transducer. The intraventricular balloon is inflated to provide a systolic pressure of 80-100 mmHg, and a diastolic pressure ≦10 mmHg. Total coronary flow is also continuously monitored using an in-line flow probe and normalized for heart weight;

The heart is allowed to equilibrate for 30 min, over which time the heart must show stable left ventricular pressures within the parameters outlined above. If the heart rate falls below 180 bpm at any time prior to the 30 min period of regional ischemia, the heart is paced at about 200 bpm for the remainder of the experiment. Ischemic preconditioning is induced by total cessation of cardiac perfusion (global ischemia) for 5 min, followed by reperfusion for 10 min. The global ischemia/reperfusion is repeated one additional time, followed by a 30 min regional ischemia. The regional ischemia is provided by tightening the snare around the coronary artery branch. Following the 30 min regional ischemia, the snare is released and the heart reperfused for an additional 120 min.

Pharmacological cardioprotection is induced by infusing the test compound at predetermined concentrations, starting 30 min prior to the 30 min regional ischemia, and continuing until the end of the 120 min reperfusion period. Hearts, which receive test compound, do not undergo the two periods of ischemic preconditioning. The reference compound, APNEA (500 nM) is perfused through hearts (which do not receive the test compound) for a 5 min period which ends 10 minutes before the 30 minute regional ischemia.

At the end of the 120 minute reperfusion period, the coronary artery snare is tightened, and a 0.5% suspension of fluorescent zinc cadmium sulfate particles (1-10 μm) is perfused through the heart; this stains all of the myocardium, except that area at risk for infarct development (area-at-risk). The heart is removed from the Langendorff apparatus, blotted dry, weighed, wrapped in aluminum foil and stored overnight at −20° C. The next day, the heart is sliced into 2 mm transverse sections from the apex to just above the coronary artery snare. The slices are stained with 1% triphenyl tetrazolium chloride (TTC) in phosphate-buffered saline for 20 min at 37° C. Since TTC reacts with living tissue (containing NAD-dependent dehydrogenases), this stain differentiates between living (red stained) tissue, and dead tissue (unstained infarcted tissue). The infarcted area (no stain) and the area-at-risk (no fluorescent particles) are calculated for each slice of left ventricle using a precalibrated image analyzer. To normalize the ischemic injury for difference in the area-at-risk between hearts, the data is expressed as the ratio of infarct area vs. area-at-risk (%IA/AAR).

The activity and thus utility of the compounds of the present invention as medical agents in providing protection from ischemic damage to tissue in a mammal can be further demonstrated by the activity of the compounds in the in vitro assay described hereinbelow. The assay also provides a means whereby the activities of the compounds of this invention can be compared with the activities of other known compounds. The results of these comparisons are useful for determining dosage levels in mammals, including humans, for inducing protection from ischemia.

The activity of an aldose reductase inhibitor in a tissue can be determined by testing the amount of aldose reductase inhibitor that is required to inhibit tissue sorbitol or lower tissue fructose (by inhibiting its production from sorbitol consequent to blocking aldose reductase). While not wishing to be bound by any particular theory or mechanism, it is believed that an aldose reductase inhibitor, by inhibiting aldose reductase, prevents or reduces ischemic damage as described hereinafter in the following paragraph.

When the supply of oxygenated blood to a tissue is interrupted or slowed down (ischemia) the cells in the oxygen-deficient tissue derive their energy (ATP) from glucose via glycolysis (which does not require the presence of oxygen). Glycolysis also requires a supply of NAD⁺ and in an ischemic tissue the length of time glycolysis can be maintained becomes sensitive to the supply of NAD⁺. Thus, it follows that sparing NAD⁺ use by aldose reductase inhibitors will enhance or prolong the.ability of ischemic tissue to carry out glycolysis, i.e., to produce energy in the absence of oxygen and in turn enhance and prolong the survival of the cells in the tissue. Since, inhibition of aldose reductase will retard depletion of the tissue's NAD⁺, an aldose reductase inhibitor is an effective anti-ischemic agent.

One aspect of this invention relates to pharmaceutical compositions comprising a compound of formula I and/or a compound of formula II of this invention and a cyclooxygenase-2 (COX-2) inhibitor. This invention also relates to therapeutic methods for treating or preventing diabetic complications in a mammal wherein a compound of formula I and/or a compound of formula II of this invention and a cyclooxygenase-2 inhibitor are administered together. The therapeutic methods of this invention include methods wherein a compound of formula I and/or a compound of formula II of this invention and a cyclooxygenase-2 inhibitor are administered together as part of the same pharmaceutical composition and to methods wherein these two agents are administered separately, either simultaneously or sequentially in any order. This invention further provides pharmaceutical kits comprising a compound of formula I and/or compounds of formula II of this invention and a cyclooxygenase-2 inhibitor.

The compounds of formula I and formula II of the composition, method and kit aspects of the present invention inhibit the bioconversion of glucose to sorbitol catalyzed by the enzyme aldose reductase and as such have utility in the treatment of diabetic complications including but not limited to such complications as diabetic neuropathy, diabetic nephropathy, diabetic cardiomyopathy, diabetic retinopathy, diabetic cataracts and tissue ischemia. Such aldose reductase inhibition is readily determined by those skilled in the art according to standard assays known to those skilled in the art (e.g., B. L. Mylari, et al., J. Med. Chem., 1991, 34, 108-122) and according to the protocol described in the General Experimental Procedures.

In the therapeutic method aspects of this invention the compounds of formula I and/or compounds of formula II of this invention are administered together with a cyclooxygenase-2 inhibitor as part of an appropriate dosage regimen designed to obtain the benefits of the therapy. With respect to the compounds of formula I and formula II, the appropriate dosage regimen, the amount of each dose administered and the intervals between doses of the compound will depend upon the compound of formula I and/or formula II of this invention being used, the type of pharmaceutical compositions being used, the characteristics of the subject being treated and the severity of the conditions. Generally, in carrying out the methods of this invention, an effective dosage for the compounds of formula I and formula II of this invention is in the range of about 0.05 mg/kg/day to about 500 mg/kg/day in single or divided doses. For human administration a preferred dosage is about 5 mg to about 500 mg per subject per day. However, some variation in dosage will necessarily occur depending on the condition of the subject being treated. The individual responsible for dosing will, in any event, determine the appropriate dose for the individual subject.

The standard assays used to determine aldose reductase inhibiting activity, as described above, may be used to determine dosage levels in humans and other mammals of the compounds of formula I and formula II of this invention. Such assays provide a means to compare the activities of the compounds of formula I and formula II of this invention and other known compounds that are aldose reductase inhibitors. The results of these comparisons are useful for determining such dosage levels.

Any cyclooxygenase-2 (COX-2) inhibitor may be used in this invention. The term selective cyclooxygenase-2 inhibitor refers to a pharmaceutical agent that selectively inhibits the enzyme cyclooxygenase-2. The following patents and patent applications exemplify cyclooxygenase-2 inhibitors which can be used in the combination compositions, methods and kits of this invention, and refer to methods of preparing those cyclooxygenase-2 inhibitors: U.S. Pat. No. 5,817,700; PCT application publication WO97/28121; U.S. Pat. Nos. 5,767,291; 5,436,265; 5,474,995; 5,536,752; 5,550,142; 5,604,260; 5,698,584; 5,710,140; 5,840,746; Great Britain Patent Application 986430; PCT application publication WO97/28120; Great Britain Patent Application 9800689; Great Britain Patent Application 9800688; PCT application publication WO94/14977; PCT application publication WO98/43966; PCT application publication WO98/03484; PCT application publication WO98/41516; PCT application publication WO98/41511; Great Britain Patent Application 2,319,032; PCT application publication WO96/37467; PCT application publication WO96/37469; PCT application publication WO96/36623; PCT application publication WO98/00416; PCT application publication WO97/44027; PCT application publication WO97/44028; PCT application publication WO96/23786; PCT application publication WO97/40012; PCT application publication WO96/19469; PCT application publication WO97/36863; PCT application publication WO97/14691; PCT application publication WO97/11701; PCT application publication WO96/13483; PCT application publication WO96/37468; PCT application publication WO96/06840; PCT application publication WO94/26731; PCT application publication WO94/20480; U.S. Pat. Nos. 5,006,549; 4,800,211; 4,782,080; 4,720,503; 4,760,086; 5,068,248; 5,859,257; PCT application publication WO98/47509; PCT application publication WO98/47890; PCT application publication WO98/43648; PCT application publication WO98/25896; PCT application publication WO98/22101; PCT application publication WO98/16227; PCT application publication WO98/06708; PCT application publication WO97/38986; U.S. Pat. No. 5,663,180; PCT application publication WO97/29776; PCT application publication WO97/29775; PCT application publication WO97/29774; PCT application publication WO97/27181; PCT application publication WO95/11883; PCT application publication WO97/14679; PCT application publication WO97/11704; PCT application publication WO96/41645; PCT application publication WO96/41626; PCT application publication WO96/41625; PCT application publication WO96/38442; PCT application publication WO96/38418; PCT application publication WO96/36617; PCT application publication WO96/24585; PCT application publication WO96/24584; PCT application publication WO96/16934; PCT application publication WO96/03385; PCT application publication WO96/12703; PCT application publication WO96/09304; PCT application publication WO96/09293; PCT application publication WO96/03392; PCT application publication WO96/03388; PCT application publication WO96/03387; PCT application publication WO96/02515; PCT application publication WO96/02486; U.S. Pat. No. 5,476,944; PCT application publication WO95/30652; U.S. Pat. No. 5,451,604; PCT application publication WO95/21817; PCT application publication WO95/21197; PCT application publication WO95/15315; U.S. Pat. Nos. 5,504,215; 5,508,426; 5,516,907; 5,521,207; 5,753,688; 5,760,068; 5,420,343; PCT application publication WO95/30656; U.S. Pat. No. 5,393,790; and PCT application publication WO94/27980, published Feb. 8, 1994. The foregoing patents and patent applications are wholly incorporated herein by reference.

Preferred cyclooxygenase-2 inhibitors which may be used in accordance with this invention include celecoxib, also known as Celebrex®, and rofecoxib, also known as Vioxx® and etoricoxib,

The activity of the cyclooxygenase-2 inhibitors of the present invention may be evaluated using the human cell based assay described in Moore et al., Inflam. Res., 45, 54, 1996. Activity may also be evaluated by the in vivo carrageenan induced foot edema rat study described in Winter et al., Proc. Soc. Exp. Biol. Med., 111, 544,1962.

Cyclooxygenase-2 inhibitors are preferably administered in amounts ranging from about 0.01 mg/kg/day to 500 mg/kg/day in single or divided doses, preferably about 10 mg/kg/day to about 300 mg/kg/day for an average subject, depending upon the cyclooxygenase-2 inhibitor and the route of administration. However, some variation in dosage will necessarily occur depending on the condition of the subject being treated. The person responsible for administration will, in any event, determine the appropriate dose for the individual subject.

In the aspects of this invention related to therapeutic methods of treating or preventing diabetic complications wherein a compound of formula I and/or a compound of formula II and a cyclooxygenase-2 inhibitor are administered together as part of the same pharmaceutical composition and to methods wherein these two agents are administered separately, the appropriate dosage regimen, the amount of each dose administered and the intervals between doses of the active agents will again depend upon the compound of formula I and/or formula II and the cyclooxygenase-2 inhibitor being used, the type of pharmaceutical compositions being used, the characteristics of the subject being treated and the severity of the condition(s).

Administration of the compounds and pharmaceutical compositions of this invention may be performed via any method which delivers a compound or composition of this invention preferentially to the desired tissue (e.g., nerve, kidney, lens, retina and/or cardiac tissues). These methods include oral routes, parenteral, intraduodenal routes, by inhalation, etc., and may be administered in single (e.g., once daily) or multiple doses or via constant infusion.

The pharmaceutical compositions of this invention may be administered to a subject in need of treatment by a variety of conventional routes of administration, including orally, topically, parenterally, e.g., intravenously, rectally, subcutaneously or intramedullar. Further, the pharmaceutical compositions of this invention may be administered intranasally, as a suppository or using a “flash” formulation, i.e., allowing the medication to dissolve in the mouth without the need to use water.

The compounds of this invention may be administered alone or in combination with pharmaceutically acceptable carriers, vehicles or diluents, in either single or multiple doses. Suitable pharmaceutical carriers, vehicles and diluents include inert solid diluents or fillers, sterile aqueous solutions and various organic solvents. The pharmaceutical compositions formed by combining the compounds of this invention and the pharmaceutically acceptable carriers, vehicles or diluents are then readily administered in a variety of dosage forms such as tablets, powders, lozenges, syrups, injectable solutions and the like. These pharmaceutical compositions can, if desired, contain additional ingredients such as flavorings, binders, excipients and the like. Thus, for purposes of oral administration, tablets containing various excipients such as sodium citrate, calcium carbonate and/or calcium phosphate may be employed along with various disintegrants such as starch, alginic acid and/or certain complex silicates, together with binding agents such as polyvinylpyrrolidone, sucrose, gelatin and/or acacia. Additionally, lubricating agents such as magnesium stearate, sodium lauryl sulfate and talc are often useful for tabletting purposes. Solid compositions of a similar type may also be employed as fillers in soft and hard filled gelatin capsules. Preferred materials for this include lactose or milk sugar and high molecular weight polyethylene glycols. When aqueous suspensions or elixirs are desired for oral administration, the active pharmaceutical agent therein may be combined with various sweetening or flavoring agents, coloring matter or dyes and, if desired, emulsifying or suspending agents, together with diluents such as water, ethanol, propylene glycol, glycerin and/or combinations thereof.

For parenteral administration, solutions of the compounds of this invention in sesame or peanut oil, aqueous propylene glycol, or in sterile aqueous solutions may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, the sterile aqueous media employed are all readily available by standard techniques known to those skilled in the art.

Generally, a composition of this invention is administered orally, or parenterally (e.g., intravenous, intramuscular, subcutaneous or intramedullary). Topical administration may also be indicated, for example, where the patient is suffering from gastrointestinal disorders or whenever the medication is best applied to the surface of a tissue or organ as determined by the attending physician.

Buccal administration of a composition of this invention may take the form of tablets or lozenges formulated in a conventional manner.

For intranasal administration or administration by inhalation, the compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of a compound of this invention. Capsules and cartridges (made, for example, from gelatin) for use in an inhaler or insufflator may be formulated containing a powder mix of a compound or compounds of the invention and a suitable powder base such as lactose or starch.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easton, Pa., 19th Edition (1995).

In the composition aspects of this invention, wherein the compositions contain an amount of both a first compound selected from a compound of formula I and a compound of formula II of this invention and a second compound that is a cyclooxygenase-2 inhibitor, the amount of each such ingredient may independently be, 0.0001%-95% of the total amount of the composition, provided, of course, that the total amount does not exceed 100%. In any event, the composition or formulation to be administered will contain a quantity of each of the components of the composition according to the invention in an amount effective to treat the disease/condition of the subject being treated.

Since the present invention has an aspect that relates to the treatment of the disease/conditions described herein with a combination of active ingredients which may be administered separately, the invention also relates to combining separate pharmaceutical compositions in kit form. The kit comprises two separate pharmaceutical compositions: a first pharmaceutical composition comprising a compound of formula I and/or a compound of formula II of this invention; and a second pharmaceutical composition comprising a cyclooxygenase-2 inhibitor. The kit also comprises a container for containing the separate compositions such as a divided bottle or a divided foil packet. Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc . . . Second Week, Monday, Tuesday, . . . ” etc. Other variations of memory aids will be readily apparent. A “daily dose” can be a single tablet or capsule or several tablets or capsules to be taken on a given day. Also, a daily dose of a compound of Formula I or Formula II of this invention can consist of one tablet or capsule while a daily dose of the cyclooxygenase-2 inhibitor can consist of several tablets or capsules, or vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The journal articles and scientific references, patents and patent application publications cited above are wholly incorporated herein by reference.

### GENERAL EXPERIMENTAL PROCEDURES

Melting points were determined on a Thomas-Hoover capillary melting point apparatus, and are uncorrected. ¹H NMR spectra were obtained on a Bruker AM-250 (Bruker Co., Billerica, Mass.), a Bruker AM-300, a Varian XL-300 (Varian Co., Palo Alto, Calif.), or a Varian Unity 400 at about 23° C. at 250, 300, or 400 MHz for proton. Chemical shifts are reported in parts per million (6) relative to residual chloroform (7.26 ppm), dimethylsulfoxide (2.49 ppm), or methanol (3.30 ppm) as an internal reference. The peak shapes and descriptors for the peak shapes are denoted as follows: s, singlet; d, doublet; t, triplet; q, quartet; m, multiplet; c, complex; br, broad; app, apparent. Low-resolution mass spectra were obtained under thermospray (TS) conditions on a Fisons (now Micromass) Trio 1000 Mass Spectrometer (Micromass Inc., Beverly, Mass.), under chemical-ionization (cl) conditions on a Hewlett Packard 5989A Particle Beam Mass Spectrometer (Hewlett Packard Co., Palo Alto, Calif.), or under atmospheric pressure chemical ionization (APCl) on a Fisons (now Micromass) Platform II Spectrometer.

### EXAMPLE 1

### 6-(Indole-2-sulfonyl)-2H-pyridazin-3-one

Step A: 3-Methoxy-6-(indole-2-sulfenyl)-pyridazine. To a solution of 2-mercaptoindole (6.7 mmol, 1.0 g) in acetone (20 mL) was added 2-chloro-6-methoxy-pyridazine (144 mmol, 1.52 g) and potassium carbonate (70 mmol, 0.98 g) and the reaction mixture was refluxed for 2 hours. Excess acetone was removed and the residue was partitioned between CHCl₃ (20 mL) and H₂O (20 mL). The CHCl₃ layer was collected, dried, filtered and the filtrate was evaporated to a residue, which was purified by silica gel chromatography (eluent: hexanes:EtOAc::4:1) to obtain 3-methoxy-6-(indole-2-sulfenyl)-pyridazine (31%, 534 mg).

Step B: 3-Methoxy-6-(indole-2-sulfonyl)-pyridazine. To a solution of 3-methoxy-6-(indole-2-sulfenyl)-pyridazine (1.9 mmol, 488 mg) in CHCl₃ (20 mL) was added meta-chloroperbenzoic acid (MCPBA, 4.1 mmol, 1.0 g) and the reaction mixture was stirred overnight at room temperature. The reaction mixture was filtered and the filtrate was washed with saturated sodium bicarbonate solution (20 mL) and H₂O (20 mL). The chloroform layer was collected, filtered, dried and the filtrate was evaporated to a residue, which was purified by silica gel chromatography (eluent: hexanes:EtOAc::3:1) to obtain the desired product, 3-methoxy-6-(indole-2-sulfonyl)-pyridazine (33%, 180 mg).

Step C: 6-(Indole-2-sulfonyl)-2H-pyridazin-3-one. A mixture of 3-methoxy-6-(indole-2-sulfonyl)-pyridazine (0.58 mmol, 290 mg), conc. HCl (0.5 mL), and dioxane (3 mL) was heated at 100° C. for 2 hours. The reaction mixture was cooled and evaporated to dryness. Water (10 mL) was added to the residue, and the resulting solid, 6-(indole-2-sulfonyl)-2H-pyridazin-3-one was collected and dried (83%, 133 mg); mp 248° C. −249° C.

### EXAMPLE 2

### 6-(5-Chloro-3-methyl-benzofuran-2-sulfonyl)-2H-pyridazin-3-one

Step A: 5-Chloro-2-mercapto-3-methyl benzofuran. n-Butyl lithium (2.5 M in hexane, 0.09 mol, 33 mL) was added dropwise over 15 minutes to a solution of 5-chloro-3-methylbenzofuran (which was prepared as described in J. Chem. Soc., 1965, 744-777, 0.09 mol, 369 mg) in tetrahydrofuran (THF, 160 mL) cooled to −78° C. To this was added sulfur powder (0.09 mol, 2.7 g) and the reaction mixture was stirred for 10 minutes. The reaction mixture was allowed to come to room temperature and was then quenched with ether (200 mL) and H₂O (500 mL). Sufficient 10% HCl was added to adjust the pH to 7. The ether layer was collected, dried, filtered and the filtrate was evaporated to dryness to obtain a pale yellow solid, 5-chloro-2-mercapto-3-methyl benzofuran (90%, 15.1 g).

Step B: 3-(5-Chloro-3-methyl-benzofuran-2-ylsulfenyl)-6-methoxy-pyridazine. To a solution containing 5-chloro-2-mercapto-3-methyl benzofuran (10 mmol, 1.98 g and 3-chloro-6-methoxy pyridazine (10 mmol, 1.44 g) in dimethylformamide (DMF, 10 mL) was added potassium carbonate (20 mmol, 2.76 g) and the reaction mixture was stirred at room temperature for 3 hours. The reaction mixture was quenched with H₂O (200 mL), the precipitated yellow solid was collected and the solid was purified by silica gel chromatography (eluent: hexanes:EtOAc::9:1) to obtain 3-(5-chloro-3-methyl-benzofuran-2-ylsulfenyl)-6-methoxy-pyridazine (93%, 2.87 g); mp 131° C. −134° C.

Step C: 6-(5-Chloro-3-methyl-benzofuran-2-sulfenyl)-2-H-pyridazin-3-one. A mixture of 3-(5-chloro-3-methyl-benzofuran-2-ylsulfenyl)-6-methoxy-pyridazine (1.6 mmol, 500 mg), conc. HCl (1 mL), and dioxane (5 mL) was heated at 100° C. for 2 hours. The reaction mixture was cooled and evaporated to dryness. Water (10 mL) was added to the residue, and the resulting white precipitate was collected and crystallized from ethanol to obtain the desired product, 6-(5-chloro-3-methyl-benzofuran-2-sulfenyl)-2-H-pyridazin-3-one (73%, 113 mg); mp >240° C.

Step D: 6-(5-Chloro-3-methyl-benzofuran-2-sulfonyl)-2-H-pyridazin-3-one. To a mixture of 6-(5-chloro-3-methyl-benzofuran-2-sulfenyl)-2-H-pyridazin-3-one, and acetic acid (30 mL) was added peracetic acid (33 mmol, 7.8 mL). The reaction mixture was allowed to stir overnight and the precipitated solid was collected and washed with H₂O. The solid was air dried and crystallized from methanol to give 6-(5-chloro-3-methyl-benzofuran-2-sulfonyl)-2H-pyridazin-3-one, (37%, 1.81 g). mp 247° C.-248° C.

### EXAMPLE 3

### 6-(5-Chloro-3-methyl-benzofuran-2-sulfonyl)-2H-pyridazin-3-one

Step A: 3-Methoxy-6-(5-chloro-3-methyl-benzofuran-2-sulfonyl)-pyridazine. n-Butyl lithium (2.5 M in hexane, 1.2 mmol, 0.48 mL) was added dropwise over 15 minutes to a solution of 5-chloro-2-methyl benzofuran (which was prepared as described in J. Chem. Soc., 1965, 744-777, 1.92 mmol, 369 mg) in THF (6 mL) cooled to −78° C. To this was added 2-fluorosulfonyl-4-methoxy-pyridazine (1.92 mmol, 320 mg) and was stirred for 30 minutes. The reaction mixture was allowed to come to room temperature overnight and then quenched with EtOAc (20 mL) and H₂O (10 mL). The organic portion was collected, dried, filtered and the filtrate was evaporated to dryness to obtain a crude product, which was purified by silica gel chromatography (eluent: hexanes:EtOAc::3:2) to obtain the desired product: 3-methoxy-6-(5-chloro-3-methyl-benzofuran-2-sulfonyl)-pyridazine (22%, 166 mg).

Step B: 6-(3-Methyl-benzofuran-2-sulfonyl)-2H-pyridazin-3-one. A mixture of 3-methoxy-6-(5-chloro-3-methyl-benzofuran-2-sulfonyl)-pyridazine (0.5 mmol, 162 mg), conc. HCl (1 mL), and dioxane (3 mL) was heated at 100° C. for 2 hours. The reaction mixture was cooled and evaporated to dryness. Water (10 mL) was added to the residue. The resulting yellow precipitate was collected and crystallized from ethanol to obtain the desired product: 6-(3-methyl-benzofuran-2-sulfonyl)-2H-pyridazin-3-one (73%, 113 mg); mp 247° C.-248° C.

### EXAMPLE 4

### 6-(5-Chloro-3-methyl-benzofuran-2-sulfonyl)-2H-pyridazin-3-one

Step A: 3-Methoxy-6-(5-chloro-3-methyl-benzofuran-2-sulfonyl)-pyridazine. n-Butyl lithium (2.5 M in hexane, 33 mmol, 13.2 mL) was added dropwise over 15 minutes to a solution of 5-chloro-2-methyl benzofuran (which was prepared as described in J. Chem. Soc., 1965, 744-777, 1.92 mmol, 369 mg) in THF (30 mL) cooled to from between −50° C. to −35° C. This was transferred into a cold-jacketed addition funnel and added drop-wise to a solution of 3-fluorosulfonyl-6-methoxypyridazine (30 mmol, 5.76 g) in THF (30 mL) over 10 minutes. The reaction mixture was allowed to come to room temperature, excess solvents were removed, and the residue was quenched with H₂O (500 mL). The granulated solid was filtered and air dried to obtain 3-methoxy-6-(5-chloro-3-methyl-benzofuran-2-sulfonyl)-pyridazine (75%, 7.62 g).

Step B: 6-(5-Chloro-3-methyl-benzofuran-2-sulfonyl)-2H-pyridazin-2H-pyridazin-3-one. A mixture of 3-methoxy-6-(5-chloro-3-methyl-benzofuran-2-sulfonyl)-pyridazine (22.2 mmol, 7.5 g), conc. HCl (5 mL), and dioxane (50 mL) was heated at 100° C. for 2 hours. The reaction mixture was cooled and evaporated to dryness. Water (20 mL) was added to the residue. The resulting precipitate was collected and crystallized from ethanol to obtain the desired product: 6-(5-chloro-3-methyl-benzofuran-2-sulfonyl)-2H-pyridazin-2H-pyridazin-3-one (89%, 6.42 g).

### EXAMPLE 5

### 6-(Benzofuran-2-sulfonyl)-2H-pyridazin-3-one

The title compound of Example 5 was prepared from benzofuran in a manner analogous to the method of Example 3. (10%); mp 210° C.-211° C.

### EXAMPLE 6

### 6-(5-Methoxy-benzofuran-2-sulfonyl)-2H-pyridazin-3-one

The title compound of Example 6 was prepared from 5-methoxybenzofuran in a manner analogous to the method of Example 3. (28%); mp 222° C.-223° C.

### EXAMPLE 7

### 6-(3,5-Dimethyl-benzofuran-2-sulfonyl)-2H-pyridazin-3-one

The title compound of Example 7 was prepared from 3,5-dimethylbenzofuran in a manner analogous to the method of Example 3. (68%); mp 246° C.-247° C.

### EXAMPLE 8

### 6-(5,7-Dichloro-benzofuran-2-sulfonyl)-2H-pyridazin-3-one

The title compound of Example 8 was prepared from 5,7-dichloro-benzofuran in a manner analogous to the method of Example 3. mp 240° C.-245° C.

### EXAMPLE 9

### 6-(5-Chloro-benzofuran-2-sulfonyl)-2H-pyridazin-3-one

The title compound of Example 9 was prepared from 5-chlorobenzofuran in a manner analogous to the method of Example 5. (68%); mp 246-247° C.

### EXAMPLE 10

### 6-(4-Chloro-3-methyl-benzofuran-2-sulfonyl)-2H-pyridazin-3-one

The title compound of Example 10 was prepared from 4-chloro-3-methyl benzofuran in a manner analogous to the method of Example 5. (25%, mp 232° C.-233° C.).

### EXAMPLE 11

### 6-(3-Methyl-benzofuran-2-sulfonyl)-2H-pyridazin-3-one

Step A: 3-Methoxy-6-(3-methyl-benzofuran-2-sulfonyl)-pyridazine. A solution of 2-bromo-3-methyl benzofuran (Helv. Chim. Acta, 1948, 31, 78) (1.34 mmol, 283 mg) in THF (5 mL) was cooled to −78° C. and n-butyl lithium (2.5 M in hexane, 1.47 mmol, 0.6 mL) was added dropwise. The reaction mixture was stirred for 30 minutes and 2-fluorosulfonyl-4-methoxy-pyridazine (1.34 mmol, 257 mg) was added. The reaction mixture was allowed to come to room temperature overnight and was diluted with EtOAc (20 mL) and H₂O (10 mL). The organic portion was collected, dried, filtered and the filtrate was evaporated to dryness to obtain a brown oil, 3-methoxy-6-(3-methyl-benzofuran-2-sulfonyl)-pyridazine (52%, 212 mg).

Step B: 6-(3-Methyl-benzofuran-2-sulfonyl)-2H-pyridazin-3-one. A mixture of the above product (0.73 mmol, 212 mg), conc. HCl (2 mL), and dioxane (3 mL) was heated at 100° C. for 2 hours. The reaction mixture was cooled and evaporated to dryness to obtain a crude product, which was purified by silica gel chromatography (eluent: EtOAc:hexanes::1:1), to obtain 6-(3-methyl-benzofuran-2-sulfonyl)-2H-pyridazin-3-one (31%, 65 mg); mp 182° C.-183° C.

### EXAMPLE 12

### 6-(5-Trifluoromethyl-3-methyl-benzofuran-2-sulfonyl)-2H-pyridazin-3-one

Step A: α,α,α-Trifluoro-o-iodo-p-cresol. A mixture of iodine (91.6 mmol, 23.2 g) and sodium bicarbonate (91.6 mmol, 7.7 g) was added to a solution of α,α,α-trifluoro-p-cresol (83.3 mmol, 13.5 g) in THF (90 mL) and H₂O (90 mL) and the reaction mixture was allowed to stand at room temperature overnight. Sufficient thiourea (5% solution) was added to remove the excess iodine as indicated by the color change of the reaction from deep violet to brown. The reaction mixture was extracted with ether (3×100 mL), the extract was dried, filtered and the filtrate was concentrated to obtain a brown oil. This oil was distilled (bp 105° C. at 44 mm Hg) to obtain α,α,α-trifluoro-o-iodo-p-cresol (4.1 g, 75% pure, admixed with the starting α,α,α-trifluoro-p-cresol).

Step B: To a mixture of the above 75% pure α,α,α-trifluoro-o-iodo-p-cresol (4.1 g, 17 mmol), potassium carbonate (7.7 g), and DMF (120 mL) was added allyl bromide (6.8 g). After 3 hours the reaction mixture was poured into H₂O (100 mL) and extracted with ether (2×100 mL). The ether layer was collected, dried, filtered and the filtrate was concentrated to obtain a brown oil. This oil was distilled (bp, 95-100° C. at 20 mm Hg) to obtain a mixture (3:1) of allyl compounds.

Step C: 3-Methyl-5-trifluoromethyl benzofuran. To a mixture of the above allyl compounds (3.9 g, 8.83 mmol of the desired isomer), sodium carbonate (22.1 mmol, 2.3 g), sodium formate (8.83 mmol, 0.81 g), n-butyl ammonium chloride (9.72 mmol, 2.7 g) and DMF (15 mL) was added palladium di-acetate (0.44 mmol, 0.1 g). The reaction mixture was heated to 80° C. and maintained at that temperature overnight. The reaction mixture was cooled to room temperature, filtered and the filtrate was dried and evaporated to give a crude product, which was purified by silica gel chromatography (eluent: hexanes) to obtain 3-methyl-5-trifluoromethyl benzofuran as a clear oil (44%, 780 mg).

Step D: 3-Methoxy-6-(5-trifluoromethyl-3-methyl-benzofuran-2-sulfonyl)-pyridazine. n-Butyl lithium (2.5 M in hexane, 4.2 mmol, 1.7 mL) was added dropwise over 15 minutes to a solution of 3-methyl-5-trifluoromethyl benzofuran (3.82 mmol, 765 mg) in THF (10 mL) cooled to −78° C. To this was added 2-fluorosulfonyl-4-methoxy-pyridazine (3.82 mmol, 734 mg) and stirred for 30 minutes. The reaction mixture was allowed to come to room temperature overnight and then quenched with EtOAc (20 mL) and H₂O (10 mL). The organic portion was collected, dried, filtered and the filtrate was evaporated to dryness to obtain a crude product, which was purified by silica gel chromatography (eluent: hexanes:EtOAc::3:1) to obtain the desired product, 3-methoxy-6-(5-trifluoromethyl-3-methyl-benzofuran-2-sulfonyl)-pyridazine (35%, 501 mg).

Step E: 6-(5-Trifluoromethyl-3-methyl-benzofuran-2-sulfonyl)-2H-pyridazin-3-one. A mixture of 3-methoxy-6-(5-trifluoromethyl-3-methyl-benzofuran-2-sulfonyl)-pyridazine (1.34 mmol, 500 mg), conc. HCl (2 mL), and dioxane (4 mL) was heated at 100° C. for 2 hours. The reaction mixture was cooled and evaporated to dryness. Water (10 mL) was added to the residue. The resulting white solid was collected and air dried to obtain the desired product: 6-(5-trifluoromethyl-3-methyl-benzofuran-2-sulfonyl)-2H-pyridazin-3-one (56%, 270 mg); mp 244° C.-245° C.

### EXAMPLE 13

### 6-(5-Chloro-3-isopropyl-benzofuran-2-sulfonyl)-2H-pyridazin-3-one

Step A: 3-Methoxy-6-(5-chloro-3-isopropyl-benzofuran-2-sulfonyl)-pyridazine. n-Butyl lithium (2.5 M in hexane, 4.04 mmol, 1.62 mL) was added dropwise over 15 minutes to a solution of 5-chloro-3-isopropyl benzofuran (which was prepared as described in J. Am. Chem. Soc., 1950, 72, 5308,3.67 mmol, 715 mg) in THF (10 mL) cooled to −78° C. To this was added 2-fluorosulfonyl-4-methoxy-pyridazine (3.67 mmol, 706 mg) and the reaction mixture was stirred for 30 minutes. The reaction mixture was allowed to come to room temperature overnight and then quenched with EtOAc (20 mL) and H₂O (10 mL). The organic portion was collected, dried, filtered and the filtrate was evaporated to dryness to obtain a crude product, which was purified by silica gel chromatography (eluent: hexanes:EtOAc::4:1) to obtain the desired product: 3-methoxy-6-(5-chloro-3-isopropyl-benzofuran-2-sulfonyl)-pyridazine (21%, 283 mg).

Step B: 6-(5-Chloro-3-isopropyl-benzofuran-2-sulfonyl)-2H-pyridazin-3-one. A mixture of the above product (0.77 mmol, 283 mg), conc. HCl (1.5 mL), and dioxane (3 mL) was heated at 100° C. for 2 hours. The reaction was cooled and evaporated to dryness. The dried residue was triturated with water (10 mL), and filtered to obtain the desired product, 6-(5-chloro-3-isopropyl-benzofuran-2-sulfonyl)-2H-pyridazin-3-one. (79%, 215 mg); mp 211° C.-212° C.

### EXAMPLE 14

### 6-(5-Fluoro-3-methyl-benzofuran-2-sulfonyl)-2H-pyridazin-3-one

Step A: (2-Acetyl-4-fluoro-phenoxy)-acetic acid. Chloroacetic acid (99.3 mmol, 9.4 g) was added to a suspension of 5-fluoro-2-hydroxy acetophenone (33.1 mmol, 5.1 g) in water (60 mL) containing sodium hydroxide (165.4 mmol, 6.6 g) and the reaction mixture was refluxed for 3.5 hours. The reaction mixture was cooled to room temperature, poured into a separatory funnel and the oily liquid at the bottom of the funnel was discarded. The aqueous top layer was collected, cooled to 0° C. and acidified with conc. HCl. The white precipitate was collected, and air died. The dry solid was crystallized from toluene to obtain (2-acetyl-4-fluoro-phenoxy)-acetic acid, (57%, 4.3 g).

Step B: 5-Fluoro-3-methyl benzofuran. Anhydrous sodium acetate (139.3 mmol, 11.4 g) was added to a solution of the title compound of Example 14, Step A (3.24 mmol, 1.6 g) in acetic anhydride (70 mL) and heated for 3 hours at 110° C. After cooling, the reaction mixture was poured into water (100 mL) and stirred for 1 hour. The aqueous solution was extracted with ether (2×100 mL), washed with 3% aqueous KOH (2×20 mL) and water (2×20 mL). The washed ether layer was collected, dried, filtered and the filtrate was evaporated to a brown residue, which was purified by silica gel chromatography (eluent: hexanes) to obtain the desired product, 5-fluoro-3-methyl benzofuran (59%, 1.77 mg).

Step C: 3-Methoxy-6-(5-fluoro-3-methyl-benzofuran-2-sulfonyl)-pyridazine. n-Butyl lithium (2.5 M in hexane, 11 mmol, 4.83 mL) was added dropwise over 15 minutes to a solution of 5-fluoro-3-methyl benzofuran (11 mmol, 1.65 mg) in THF (20 mL) cooled to −78° C. To this was added 3-fluorosulfonyl-6-methoxy-pyridazine (11 mmol, 2.11 g) and stirred for 30 minutes. The reaction mixture was allowed to come to room temperature overnight and was then quenched with EtOAc (40 mL) and H₂O (10 mL). The organic portion was collected, dried, filtered and the filtrate was evaporated to dryness to obtain a crude product, which was purified by silica gel chromatography (eluent: hexanes:EtOAc::4:1) to obtain the desired product: 3-methoxy-6-(5-fluoro-3-methyl-benzofuran-2-sulfonyl)-pyridazine (22%, 781 mg).

Step D: 6-(5-Fluoro-3-methyl-benzofuran-2-sulfonyl)-2H-pyridazin-3-one. A mixture of 3-methoxy-6-(5-fluoro-3-methyl-benzofuran-2-sulfonyl)-pyridazine (2.4 mmol, 775 mg), conc. HCl (1.5 mL), and dioxane (3 mL) was heated at 100° C. for 2 hours. The reaction mixture was cooled and evaporated to dryness. The dried residue was triturated with water (10 mL), and filtered to obtain the desired product, 6-(5-fluoro-3-methyl-benzofuran-2-sulfonyl)-2H-pyridazin-3-one (84%, 620 mg); mp 232° C.-233° C.

### EXAMPLE 15

### 6-(6-Chloro-3-methyl-benzofuran-2-sulfonyl)-2H-pyridazin-3-one

The title compound of Example 15 was prepared from 4-chloro-2-hydroxy acetophenone in a manner analogous to the method of Example 14. mp >240° C.

### EXAMPLE 16

### 6-(3-Hydroxy-benzofuran-2-sulfonyl)-2H-pyridazin-3-one

Step A: 3-Methoxy-6-(3-hydroxy-benzofuran-2-sulfonyl)-pyridazine. n-Butyl lithium (12 mmol, 4.7 mL) was added dropwise to a solution of diisopropyl amine (12 mmol, 1.7 mL) in THF (5 mL) at −78° C. After 10 minutes, a solution of 3-coumaranone (10 mmol, 1.92 g) in THF (10 mL) was added. The temperature was maintained at −78° C. and stirred for 10 minutes. To this was added a solution of 3-fluorosulfonyl-6-methoxy-pyridazine. The reaction mixture was brought to room temperature over one hour and quenched with ammonium chloride (1 g) and extracted with EtOAc (2×25 mL). The EtOAc extract was washed with H₂O, the organic layer was collected, dried, filtered and the filtrate was evaporated to a residue. This residue was purified by silica gel chromatography (eluent: hexanes:EtOAc::9:1) to yield 3-methoxy-6-(3-hydroxy-benzofuran-2-sulfonyl)-pyridazine (17%, 622 mg).

Step B: 6-(3-Hydroxy-benzofuran-2-sulfonyl)-2H-pyridazin-3-one. A mixture of 3-methoxy-6-(3-hydroxy-benzofuran-2-sulfonyl)-pyridazine (2.7 mmol, 820 mg), conc. HCl (2 mL), and dioxane (10 mL) was heated at 100° C. for 2 hours. The reaction mixture was cooled and evaporated to dryness. The dried residue was extracted with EtOAc (2×20 mL). The extract was dried, filtered, and the filtrate was evaporated to a residue, which was purified by silica gel chromatography (eluent: EtOAc:n-hexanes::3:1), triturated with water (10 mL), and filtered to obtain the desired product: 6-(3-hydroxy-benzofuran-2-sulfonyl)-2H-pyridazin-3-one (35%, 284 mg); mp 186° C.-189° C.

### EXAMPLE 17

### 6-(5-Chloro-3-hydroxy-benzofuran-2-sulfonyl)-2H-pyridazin-3-one

The title compound of Example 17 was prepared from from 5-chloro-3-comaranone in place of 3-comaranone in a manner analogous to the method of Example 16. (22%); mp >240° C.

### EXAMPLE 18

### 6-(5-Chloro-3-methyl-benzothiophene-2-sulfonyl)-2H-pyridazin-3-one

Step A: 3-Methoxy-6-(5-chloro-3-methyl-benzothiophene-2-sulfonyl)-pyridazine. n-Butyl lithium (2.5 M in hexane, 2.1 mmol, 0.84 mL) was added dropwise over 15 minutes to a solution of 5-chloro-3-methyl benzothiophene (1.91 mmol, 348 mg, which was prepared as described in J. Chem. Soc., 1965, 774-777), in THF (6 mL) cooled to −78° C. To this was added 2-fluorosulfonyl-4-methoxy-pyridazine (1.91 mmol, 366 mg) and stirred for 30 minutes. The reaction mixture was allowed to come to room temperature overnight and then quenched with EtOAc (20 mL) and H₂O (10 mL). The organic portion was collected, dried, filtered and the filtrate was evaporated to dryness to obtain a crude product, which was purified by silica gel chromatography (eluent: hexanes:EtOAc::4:1) to obtain the desired product, 3-methoxy-6-(5-chloro-3-methyl-benzothiophene-2-sulfonyl)-pyridazine (29%, 197 mg).

Step B: 6-(5-Chloro-3-methyl-benzothiophene-2-sulfonyl)-2H-pyridazin-3-one.

A mixture of 3-methoxy-6(5-chloro-3-methyl-benzothiophene-2-sulfonyl)-pyridazine, (0.55 mmol, 197 mg), conc. HCl (1 mL), and dioxane (3 mL) was heated at 100° C. for 2 hours. The reaction mixture was cooled and evaporated to dryness. Water (10 mL) was added to the residue and the resulting yellow precipitate, 6-(5-chloro-3-methyl-benzothiophene-2-sulfonyl)-2H-pyridazin-3-one, was collected (29%, 55 mg); mp 258° C.-259° C.

### EXAMPLE 19

### 6-(5-Methyl-benzothiophene-2-sulfonyl)-2H-pyridazin-3-one

The title compound of Example 19 was prepared from 5-methyl-benzothiophene in a manner analogous to the method of Example 18 (mp 240° C.-242° C.).

### EXAMPLE 20

### 6-(Benzothiophene-2-sulfonyl)-2H-pyridazin-3-one

The title compound of Example 20 was prepared from benzothiophene in a manner analogous to the method of Example 18. mp 209° C.-210° C.

### EXAMPLE 21

### 6-(3-Phenyl-benzofuran-2-sulfonyl)-2H-pyridazin-3-one

The title compound of Example 21 was prepared from 3-phenyl-benzofuran in a manner analogous to the method of Example 3. (65%); mp >220° C.

### EXAMPLE 22

### 6-(3-[4-Fluorophenyl]-benzofuran-2-methylsulfonyl)-2H-pyridazin-3-one

The title compound of Example 22 was prepared from 4-fluorophenyl-benzofuran in a manner analogous to the method of Example 3. mp >240° C.

### EXAMPLE 23

### 6-(Thieno[2,3b]pyridine-2-sulfonyl)-2H-pyridazin-3-one

Step A: 3-Methoxy-6-(thieno[2,3b]pyridine-2-sulfonyl)-pyridazine. n-Butyl lithium (2.5 M in hexane, 2.44 mmol, 0.97 mL) was added dropwise over 15 minutes to a solution of thieno[2,3b]pyridine (2.22 mmol, 300 mg, which was prepared according to International Patent Application Publication No. WO 005910), in THF (6 mL) cooled to −78° C. To this was added 2-fluorosulfonyl-4-methoxy-pyridazine (2.22 mmol, 426 mg) and stirred for 30 minutes. The reaction mixture was allowed to come to room temperature overnight and then quenched with EtOAc (20 mL) and H₂O (10 mL). The organic portion was collected, dried, filtered and the filtrate was evaporated to dryness to obtain a crude product, which was purified by silica gel chromatography (eluent, EtOAc) to obtain the desired product, 3-methoxy-6-(thieno[2,3b]pyridine-2-sulfonyl)-pyridazine (24%, 166 mg).

Step B: 6-(Thieno[2,3b]pyridine-2-sulfonyl)-2H-pyridazin-3-one. A mixture of 3-methoxy-6-(thieno[2,3b]pyridine-2-sulfonyl)-pyridazine, without further purification, (0.54 mmol, 166 mg), conc. HCl (1 mL), and dioxane (3 mL) was heated at 100° C. for 2 hours. The reaction mixture was cooled and evaporated to dryness. Water (10 mL) was added to the residue, and sufficient solid NaHCO₃ was added to adjust the pH to 6. It was then extracted with CHCl₃ (2×20 mL), and the CHCl₃ layer was collected, dried, filtered and the filtrate was evaporated to a residue, which was purified by silica gel chromatography (eluent: EtOAc:MeOH::9:1) to yield 6-(thieno[2,3b]pyridine-2-sulfonyl)-2H-pyridazin-3-one: (29%, 30 mg); mp 225° C.-230° C.

### EXAMPLE 23a

### 6-(Furano[2,3b]pyridine-2-sulfonyl)-2H-pyridazin-3-one

The title compound of Example 23a was prepared from furano[2,3b]pyridine in a manner analogous to the method of Example 23.

### EXAMPLE 24

### 2-(6-Oxo-1,6-dihydro-pyridazine-3-sulfonyl)-5H-furo[3,2-c]pyridin-4-one

Step A: 3-Methoxy-6-(thieno[2,3b]pyridine-4-chloro-2-sulfonyl)-pyridazine. The title compound of Example 24, Step A was prepared from 4-chloro-thieno[2,3b]pyridine (which was prepared according to the method described in International Patent Application Publication No. WO00/59510) in a manner analogous to the method of Example 23.

Step B: 2-(6-Oxo-1,6-dihydro-pyridazine-3-sulfonyl)-5H-furo[3.2-c]pyridin-4-one. A mixture of 3-methoxy-6-(thieno[2,3b]pyridine-4-chloro-2-sulfonyl)-pyridazine (0.51 mmol, 157 mg), concentrated HCl (5 mL) and dioxane (3 mL) was heated at 100° C. overnight. The reaction mixture was cooled and evaporated to dryness. Water (10 mL) was added to the residue and the precipitated solid was collected to yield 53 mg of the title compound of Example 24. (35%); mp >275° C.

### EXAMPLE 25

### 6-(5-Chloro-3-ethyl-benzofuran-2-sulfonyl)-2H-pyridazin-3-one

Step A: 4-Chloro-2-iodo phenol. To a solution of 4-chlorophenol in THF (75 mL), and H₂O (75 mL) was added a mixture of crushed iodine (78.7 mmol, 20 g) and sodium bicarbonate (78.7 mmol, 6.6 g). The reaction mixture was stirred at room temperature overnight, then quenched with sufficient 5% sodium thiosulfate solution to turn the color of the reaction mixture from deep violet to light yellow and extracted with ether (2×200 mL). The ether layer was collected, washed with H₂O , and the washed ether layer was dried, filtered and the filtrate was evaporated to a crude product, which was purified by distillation to obtain 4-chloro-2-iodo phenol (7%, 1.3 g); mp 79° C.-82° C.

Step B: 4-Chloro-2-iodo O-crotylphenol. To a mixture of 4-chloro-2-iodo phenol (5.11 mmol, 1.3 g) in DMF (40 mL) and potassium carbonate (10 mmol, 1.4 g) was added crotylbromide (10.2 mmol, 1.6 g) and the reaction mixture was stirred at room temperature for one hour. The reaction was quenched with H₂O (100 mL) and extracted with EtOAc (2×50 mL). The EtOAc layer was collected, dried, filtered and the filtrate was evaporated to obtain 4-chloro-2-iodo O-crotyl phenol (94%, 1.5 g).

Step C: 5-Chloro-3-ethyl-benzofuran. To a mixture of 4-chloro-2-iodo O-crotyl phenol (1.5 g, 4.86 mmol), sodium carbonate (12.2 mmol, 1.3 g), sodium formate (4.86 mmol, 330 mg), n-butyl ammonium chloride (5.34 mmol, 1.5 g) and DMF (10 mL) was added palladium di-acetate (0.24 mmol, 55 mg). The reaction was heated at 80° C. and maintained at that temperature overnight. After bringing the reaction to room temperature, the mixture was filtered. The filtrate was dried and evaporated to give a crude product, which was purified by silica gel chromatography (eluent: hexanes) to obtain 5-chloro-3-ethyl-benzofuran as a clear oil (60%, 530 mg).

Step D: 3-Methoxy-6-(5-chloro-3-ethyl-benzofuran-2-sulfonyl)-pyridazine. n-Butyl lithium (2.5 M in hexane, 3.2 mmol, 1.3 mL) was added dropwise over 15 minutes to a solution of 5-chloro-3-ethyl-benzofuran (2.88 mmol, 520 mg) in THF (8 mL) cooled to −78° C. To this was added 2-fluorosulfonyl-4-methoxy-pyridazine (2.88 mmol, 553 mg) and the reaction mixture was stirred for 30 minutes. The reaction mixture was allowed to come to room temperature overnight and then quenched with EtOAc (20 mL) and H₂O (10 mL). The organic portion was collected, dried, filtered and the filtrate was evaporated to dryness to obtain a crude product, which was purified by silica gel chromatography (eluent: hexanes:EtOAc::4:1) to obtain the desired product: 3-methoxy-6-(5-chloro-3-ethyl-benzofuran-2-sulfonyl)-pyridazine (35%, 352 mg).

Step E: 6-(5-Chloro-3-ethyl-benzofuran-2-sulfonyl)-2H-pyridazin-3-one. A mixture of 3-methoxy-6-(5-chloro-3-ethyl-benzofuran-2-sulfonyl)-pyridazine, without further purification, (1.04 mmol, 352 mg), conc. HCl (1.5 mL), and dioxane (3 mL) was heated at 100° C. for 2 hours. The reaction mixture was cooled and evaporated to dryness. Water (10 mL) was added to the residue and the resulting solid, 6-(5-chloro-3-ethyl-benzofuran-2-sulfonyl)-2H-pyridazin-3-one, was collected. (46%, 155 mg); mp 209° C.-210° C.

### EXAMPLE 26

### 6-(Imidazo[1,2a]pyridine-3-sulfonyl)-2H-pyridazin-3-one

Step A: 6-(Imidazo[1,2a]pyridine-3-sulfonyl)-3-methoxy-pyridazine. n-Butyl lithium (2.5 M in hexane, 5 mmol, 2 mL) was added dropwise over 15 minutes to a solution of [1,2a]imidazopyridine (5 mmol, 590 mg) in THF (10 mL) cooled to −78° C. To this was added 3-fluorosulfonyl-6-methoxy-pyridazine (5 mmol, 960 mg) and the reaction mixture was stirred for 30 minutes. The reaction mixture was allowed to come to room temperature overnight and then quenched with EtOAc (20 mL) and H₂O (10 mL). The organic portion was collected, dried, filtered and the filtrate was evaporated to dryness to obtain a crude product, which was purified by silica gel chromatography (eluent: EtOAc) to obtain the desired product: 6-(imidazo[1,2a]pyridine-3-sulfonyl)-3-methoxy-pyridazine (8%, 121 mg).

Step B: 6-(Imidazo[1,2a]pyridine-3-sulfonyl)-2H-pyridazin-3-one. A mixture of 6-(imidazo[1,2a]pyridine-3-sulfonyl)-3-methoxy-pyridazine (0.341 mmol, 100 mg), conc. HCl (0.5 mL) and dioxane (5 mL) was heated at 100° C. for two hours. The reaction mixture was cooled and evaporated to dryness. Water (10 mL) was added to the residue, the pH adjusted to 7 and the resulting solid, 6-(imidazo[1,2a]pyridine-3-sulfonyl)-2H-pyridazin-3-one, was collected (72%, 67 mg); mp >240° C.

### EXAMPLE 27

### 6-(Indole-2-sulfonyl)-2H-pyridazin-3-one

Step A: 3-Methoxy-6(N-phenylsulfonylindole-2-sulfonyl)-pyridazine. t-Butyl lithium (2.5M in hexane, 6.5 mmol, 4.3 mL) was added dropwise over 15 minutes to a solution of N-sulfonylphenyl indole (2.88 mmol, 520 mg) in tetrahydrofuran (8 mL) cooled to −78° C. To this was added 2-fluorosulfonyl-4-methoxypyridazine (5.2 mmol, 1.0 g) and stirred for 30 minutes. The reaction mixture was allowed to come to room temperature overnight and then quenched with EtOAc (20 mL) and H₂O (10 mL). The organic portion was collected, dried, filtered and the filtrate was evaporated to dryness to obtain a crude product, which was purified by silica gel chromatography (eluent: hexanes:EtOAc::7:1) to obtain the desired product: 3-methoxy-6(N-phenylsulfonylindole-2-sulfonyl)-pyridazine (39%, 867 mg).

Step B: 2-Methoxy-6(indole-2-sulfonyl)-pyridazine. To a solution of sodium metal (18.6 mmol, 428 mg) dissolved in methanol (8 mL) was added a solution of 3-methoxy-6-(N-phenylsulfonylindole-2-sulfonyl)-pyridazine (1.86 mmol, 850 mg) and the reaction was stirred for 10 minutes. The reaction mixture was quenched with H₂O (10 mL) and CHCl₃ (25 mL). The CHCl₃ layer was collected, dried, filtered, and the filtrate was evaporated to obtain 2-methoxy-6-(indole-2-sulfonyl)-pyridazine (82%, 440 mg).

Step C: 6-(Indole-2-sulfonyl)-2H-pyridazin-3-one. A mixture of 2-methoxy-6-(indole-2-sulfonyl)-pyridazine (1.03 mmol, 300 mg), conc. HCl (1 mL), and dioxane (6 mL) was heated at 100° C. for two hours. The reaction mixture was cooled and evaporated to dryness. Water (10 mL) was added to the residue and the resulting solid was triturated with methanol (2 mL) to yield 6-(indole-2-sulfonyl)-2H-pyridazin-3-one (37%, 106 mg); mp 248° C.-249° C.

### EXAMPLE 28

### 6-(6-Chloro-indole-2-sulfonyl)-2H-pyridazin-3-one

The title compound of Example 28 was prepared from 6-chloro-N-p-tolylsulfonyl indole in a manner analogous to the method of Example 27. (95%); mp >250° C.

### EXAMPLE 29

### 6-(5-Methoxy-indole-2-sulfonyl)-2H-pyridazin-3-one

The title compound of Example 29 was prepared from 5-methoxy-N-p-tolylsulfonyl indole in a manner analogous to the method of Example 27. (63%); mp >250° C.

### EXAMPLE 30

### 6-(5-Chloro-indole-2-sulfonyl)-2H-pyridazin-3-one

The title compound of Example 30 was prepared from 5-chloro-N-p-tolylsulfonyl indole in a manner analogous to the method of Example 27. (64%); mp >250° C.

### EXAMPLE 31

### 6-(6-Fluoro-indole-2-sulfonyl)-2H-pyridazin-3-one

The title compound of Example 31 was prepared from 6-fluoro-N-p-tolylsulfonyl indole in a manner analogous to the method of Example 27. (90%); mp >250° C.

### EXAMPLE 32

### 6-(5,6-Methylenedioxy-indole-2-sulfonyl)-2H-pyridazin-3-one

The title compound of Example 32 was prepared from 5,6-methylenedioxy-N-p-tolylsulfonyl indole in a manner analogous to the method of Example 27. (67%).

### EXAMPLE 33

### 6-(5,7-Dichloro-indole-2-sulfonyl)-2H-pyridazin-3-one

The title compound of Example 33 was prepared from 5,7-dichloro-N-p-tolylsulfonyl indole in a manner analogous to the method of Example 27. (80%); mp >250° C.

### EXAMPLE 34

### 6-(7-Chloro-indole-2-sulfonyl)-2H-pyridazin-3-one

The title compound of Example 34 was prepared from 7-chloro-N-p-tolylsulfonyl indole in a manner analogous to the method of Example 27. (76%); mp 248-250° C.

### EXAMPLE 35

### 6-(5-Chloro-3-phenyl-2-sulfonyl)-2H-pyridazin-3-one

The title compound of Example 35 was prepared from 5-chloro-3-phenyl-benzofuran in a manner analogous to the method of Example 27. mp >240° C.

### EXAMPLE 36

### 6-(3-Chloro-indole-2-sulfonyl)-2H-pyridazin-3-one

Step A: 3-Methoxy-6-(3-chloro-indole-2-sulfenyl)-pyridazine. A mixture of 3-methoxy-6-(indole-2-sulfenyl)-pyridazine) (2.92 mmol, 750 mg), N-chloro-succinimide (2.92 mmol, 390 mg) and methanol (15 mL) was stirred overnight at room temperature. Excess methanol was removed and the residue was extracted with EtOAc (3×10 mL). The EtOAc extract was collected, dried, filtered and evaporated to dryness to obtain a residue, which was purified by silica gel chromatography (eluent: hexanes:EtOAc::19:5) to yield 3-methoxy-6-(3-chloro-indole-2-sulfenyl)-pyridazine (40%, 338 mg).

Step B: 3-Methoxy-6-(3-chloro-indole-2-sulfonyl)-pyridazine. A mixture of 3-methoxy-6-(3-chloro-indole-2-sulfenyl)-pyridazine (0.72 mmol, 210 mg), MCPBA (1.58 mmol, 385 mg) and CHCl₃ (20 mL) was stirred overnight at room temperature. The reaction mixture was diluted with CHCl₃ (20 mL), the CHCl₃ layer was collected and washed with 2N NaOH (2×5 mL). The washed CHCl₃ layer was collected, dried, filtered, and evaporated to dryness and the residue was purified by silica gel chromatography (eluent, CHCl₃) to yield 3-methoxy-6-(3-chloro-indole-2-sulfonyl)-pyridazine.

Step C: 6-(3-Chloro-indole-2-sulfonyl)-2H-pyridazin-3-one. A mixture of 3-methoxy-6-(3-chloro-indole-2-sulfonyl)-pyridazine (0.34 mmol, 110 mg), conc. HCl (1 mL), and dioxane (3 mL) was heated at 100° C. for 2 hours. The reaction mixture was cooled and evaporated to dryness. The dried residue was triturated with water (10 mL), and filtered to obtain 6-(3-chloro-indole-2-sulfonyl)-2H-pyridazin-3-one (99%, 108 mg); mp 250° C.

### EXAMPLE 37

### 6-(N-Benzylindole-5-sulfonyl)-2H-pyridazin-3-one

Step A: 3-Methoxy-6-(N-benzylindole-5-sulfonyl)-2H-pyridazine. sec-Butyl lithium (1.3 M in hexane, 5.25 mmol, 4 mL) was added dropwise to a solution of N-benzyl-5-bromo indole (3.5 mmol, 1.0 g) in THF (5 mL) at −78° C. After 15 minutes, 2-fluorosulfonyl-4-methoxy-pyridazine (4.2 mmol, 808 mg) was added and the reaction mixture was stirred for 30 minutes. The reaction mixture was allowed to come to room temperature overnight and was then quenched with EtOAc (20 mL) and H₂O (10 mL). The organic portion was collected, dried, filtered and the filtrate was evaporated to dryness to obtain a crude product, which was purified by silica gel chromatography (eluent: hexanes:EtOAc::7:1) to obtain the desired product: 3-methoxy-6-(N-benzylindole-5-sulfonyl)-2H-pyridazine (19%, 258 mg).

Step B: 6-(N-Benzylindole-5-sulfonyl)-2H-pyridazin-3-one. A mixture of 3-methoxy-6-(N-benzylindole-5-sulfonyl)-2H-pyridazine (0.64 mmol, 245 mg), conc. HCl (0.5 mL), and dioxane (3 mL) was heated at 100° C. for 2 hours. The reaction mixture was cooled and evaporated to dryness. Water (10 mL) was added to the residue and the resulting solid, 6-(N-benzylindole-5-sulfonyl)-2H-pyridazin-3-one, was collected (55%, 102 mg).

### EXAMPLE 38

### 6-(5-Chloro-3-methyl-benzofuran-2-methylsulfonyl)-2H-pyridazin-3-one

Step A: 5-Chloro-3-methyl benzofuran-2-carboxaldehyde. n-Butyl lithium (2.5 M in hexane, 6.6 mmol, 2.6 mL) was added dropwise over 15 minutes to a solution of 5-chloro-3-methyl benzofuran (6.0 mmol, 1 g) in THF (8 mL) cooled to −78° C. To this was added DMF (12 mmol, 0.6 mL) and stirred for one hour. The reaction mixture was allowed to come to room temperature overnight and then quenched with EtOAc (20 mL) and H₂O (10 mL). The organic portion was collected, dried, filtered and the filtrate was evaporated to dryness to obtain 5-chloro-3-methyl benzofuran-2-carboxaldehyde (96%, 1.12 g), which was carried on without further purification.

Step B: 5-Chloro-3-methyl benzofuran 2-methanol. To a solution of 5-chloro-3-methyl benzofuran-2-carboxaldehyde (5.55 mmol, 1.08 g) in ethanol (25 mL) was added portion-wise sodium borohydride (16.6 mmol, 630 mg). After one hour, the ethanol was evaporated and the residue was partitioned between CHCl₃ and H₂O. The CHCl₃ layer was collected, filtered, dried, and evaporated to dryness to obtain 5-chloro-3-methyl benzofuran 2-methanol (88%, 965 mg); mp 112° C.-113° C.

Step C: 2-Bromomethyl-5-chloro-3-methyl benzofuran. A solution of 5-chloro-3-methyl benzofuran 2-methanol (18.3 mmol, 3.6 g) in ether (200 mL) was cooled to 0° C. To this was added drop-wise phosphorus tribromide (29.3 mmol, 7.9 g) and then DMF (2 mL). After allowing the reaction mixture to come to room temperature over three hours, the reaction was quenched with ice water (100 mL). The ether layer was collected, dried, filtered and the filtrate was evaporated to a yellow solid: 2-bromomethyl-5-chloro-3-methyl benzofuran (88%, 4.2 g); mp 81° C.-82° C.

Step D: 3-Methoxy-6-(3-methyl-benzofuran-2-methylsulfenyl)-pyridazine. A solution of 2-mercapto-5-methoxy pyridazine (4.33 mmol, 750 mg) in DMF (5 mL) was added dropwise to a suspension of sodium hydride (60%, 4.7 mmol, 191 mg) in DMF (5 mL) cooled to 0° C. After 10 minutes, a solution of 2-bromomethyl-5-chloro-3-methyl benzofuran (2.9 mmol, 750 mg) in DMF (5 mL) was added to the reaction mixture. After two hours, the reaction mixture was quenched with water (100 mL) and extracted with EtOAc (2×50 mL). The EtOAc layer was collected, dried, filtered and the filtrate was evaporated to obtain a yellow solid: 3-methoxy-6-(3-methyl-benzofuran-2-methylsulfenyl)-pyridazine (97%, 906 mg).

Step E: 3-Methoxy-6-(3-methyl-benzofuran-2-methylsulfonyl)pyridazine. A mixture of 3-methoxy-6-(3-methyl-benzofuran-2-methylsulfenyl)-pyridazine (2.5 mmol, 800 mg), MCPBA (75%, 7.5 mmol, 1.7 g) and CHCl₃ (20 mL) was stirred at room temperature overnight. The reaction mixture was filtered and the filtrate was washed with H₂O (50 mL), and saturated sodium bicarbonate solution (10 mL). The CHCl₃ layer was collected, dried, filtered, and evaporated to dryness to obtain 3-methoxy-6-(3-methyl-benzofuran-2-methylsulfonyl)pyridazine (96%, 850 mg).

Step F: 6-(3-Methyl-benzofuran-2-methylsulfonyl)-2H-pyridazin-3-one. A mixture of 3-methoxy-6-(3-methyl-benzofuran-2-methylsulfonyl)-pyridazine (2.4 mmol, 850 mg), conc. HCl (1.5 mL), and dioxane (3 mL) was heated at 100° C. for two hours. The reaction mixture was cooled and evaporated to dryness. Water (10 mL) was added to the residue, the resulting solid was collected and triturated with hot isopropyl ether (55%, 102 mg). The precipitated white solid, 6-(3-methyl-benzofuran-2-methylsulfonyl)-2H-pyridazin-3-one, was collected (41%, 336 mg); mp 240° C.-241° C.

### EXAMPLE 39

### 6-(Indole-3-sulfonyl)-2H-pyridazin-3-one

Step A: 3-Methoxy-6-(N-sulfonylphenyl-indole-3-sulfonyl)pyridazine. Ethyl magnesium bromide (1 M in THF, 1.8 mmol, 1.8 mL) was added to an ice cold solution of 3-iodo-N-sulfonylphenyl-indole (1.5 mmol, 575 mg, which was prepared according to Tetrahedron Letters 1998, 6849-6852) in THF (10 mL) and the reaction mixture was allowed to come to room temperature over 30 minutes. To this was added 3-fluorosulfonyl-6-methoxypyridazine (2.25 mmol, 192 mg) and the reaction mixture was stirred overnight at room temperature. The reaction mixture was quenched with H₂O (10 mL) and extracted with EtOAc (2×10 mL). The EtOAc extract was dried, filtered and the filtrate was evaporated to obtain a thick liquid, which was purified by silica gel chromatography (eluent: hexanes:EtOAc::3:1 to obtain 3-methoxy-6-(N-sulfonylphenyl-indole-3-sulfonyl)pyridazine (22%, 142 mg).

Step B: 3-Methoxy-6-(indole-3-sulfonyl)-pyridazine. To a solution of sodium metal (3 mmol, 70 mg) in methanol (1 mL) was added a solution of 3-methoxy-6-(N-sulfonylphenyl-indole-3-sulfonyl)pyridazine (0.3 mmol, 130 mg) in tetrahydrofuran (2 mL) and the reaction mixture was stirred at room temperature for 15 minutes. Cold water (5 mL) was added to the reaction mixture and extracted with ethyl acetate (2×10 mL) and the extract was dried, filtered and the filtrate was evaporated to dryness to obtain a residue, which was purified by silica gel chromatography (eluent: ethyl acetate:hexanes::1:1) to obtain 3-methoxy-6-(indole-3-sulfonyl)-pyridazine (90%); mass spectrum, m+, 289.

Step C: 6-(Indole-3-sulfonyl)-2H-pyridazin-3-one. The title compound of Example 39 was prepared from 3-methoxy-6-(indole-3-sulfonyl)pyridazine in a manner analogous to the method of Example 1. (76%); mp 248° C.-250° C.

### EXAMPLE 40

### 6-(N-Methylindole-2-sulfonyl)-2H-pyridazin-3-one

Step A: 6-(Indole-N-methyl-2-sulfonyl)-3-methoxy-pyridazine. n-Butyl lithium (2.5 M in hexane, 0.83 mmol, 0.52 mL) was added dropwise over 15 minutes to a solution of 3-methoxy-6-(indole-2-sulfonyl)-pyridazine (0.69 mmol, 200 mg) in DMF (5 mL) cooled to −30° C. Methyl iodide (1.38 mmol, 0.1 mL) was added to the solution and the reaction mixture was stirred for another 10 minutes. The reaction mixture was quenched with H₂O (10 mL) and EtOAc (20 mL) and the EtOAc layer was collected, dried and evaporated to obtain 6-(indole-N-methyl-2-sulfonyl)-3-methoxy-pyridazine as pale yellow solid (97%, 203 mg).

Step B: 6-(N-Methylindole-2-sulfonyl)-2H-pyridazin-3-one. A mixture of 6-(indole-N-methyl-2-sulfonyl)-3-methoxy-pyridazine (6.6 mmol, 303 mg), concentrated HCl (0.5 mL), and dioxane (5 mL) was heated at 10° C. for 2 hours. The reaction was cooled and evaporated to dryness. Water (10 mL) was added to the residue and the resulting solid was collected to obtain 6-(N-methylindole-2-sulfonyl)-2H-pyridazin-3-one (87%, 166 mg); mp 233° C.-235° C.

### EXAMPLE 41

### 6-(Pyrrole-1-sulfonyl)2H-pyridazin-3-one

Step A: 3-Methoxy-6-(pyrrole-1-sulfonyl)-pyridazine. To an ice-cold suspension of sodium hydride (1.86 mmol, 74 mg) in DMF (1 mL) was added a solution of pyrrole (1.86 mmol, 125 mg) in DMF (2 mL). To this was added 3-fluorosulfonyl-6-methoxypyridazine (1.55 mmol, 298 mg) and the reaction mixture was stirred overnight at room temperature. The reaction mixture was quenched with H₂O (20 mL) and EtOAc (20 mL) and the EtOAc layer was collected, dried, filtered and evaporated to a residue. The residue was purified by silica gel chromatography (eluent: hexanes:EtOAc::9:1) to obtain 3-methoxy-6-(pyrrole-1-sulfonyl)-pyridazine (30%, 112 mg).

Step B: 6-(Pyrrole-1-sulfonyl)-2H-pyridazin-3-one. A mixture of 3-methoxy-6-(pyrrole-1-sulfonyl)-pyridazine (0.46 mmol, 112 mg), conc. HCl (1 mL) and dioxane (3 mL) was heated at 100° C. for 2 hours. The reaction mixture was cooled and evaporated to dryness. Water (10 mL) was added to the residue and the resulting solid was collected to obtain 6-(pyrrole-1-sulfonyl)-2H-pyridazin-3-one (69%, 73 mg); mp 140° C.-145° C.

### EXAMPLE 42

### 6-(Imidazole-1-sulfonyl)2H-pyridazin-3-one

The title compound of Example 42 was prepared from imidazole in a manner analogous to Example 41. (73%); mp 55° C.-60° C.

### EXAMPLE 43

### 6-(Indole-1-sulfonyl)2H-pyridazin-3-one

The title compound of Example 43 was prepared from indole in a manner analogous to Example 41. (87%); mp 169-170° C.

### EXAMPLE 44

### 6-(3-Chloro-indole-1-sulfonyl)2H-pyridazin-3-one

The title compound of Example 44 was prepared from 3-chloroindole in a manner analogous to Example 41. (73%); mp >220° C.

### EXAMPLE 45

### 6-(3-Chloro-Indazole-1-sulfonyl)2H-pyridazin-3-one

The title compound of Example 45 was prepared from 3-chloro-indazole in a manner analogous to Example 41. (32%); mp 238° C.-239° C.

### EXAMPLE 46

### 6-(3-Methyl-indole-1-sulfonyl)-2H-pyridazin-3-one

The title compound of Example 46 was prepared from 3-methyl-indole in a manner analogous to Example 41. (32%); mp >220° C.

### EXAMPLE 47

### 6-(Tetrahydroquinoline-1-sulfonyl)-2H-pyridazin-3-one

Step A: 3-Methoxy-6-(tetrahydroquinoline-1-sulfonyl)-pyridazine. A mixture of 3-fluorosulfonyl-6-methoxypyridazine (2 mmol, 384 mg) and tetrahydroquinoline (4 mmol, 532 mg) was heated at 140° C. for two hours. The reaction mixture was cooled, extracted with EtOAc (20 mL), and the EtOAc extract was dried, filtered and evaporated to obtain 3-methoxy-6-(tetrahydroquinoline-1-sulfonyl)-pyridazine (73%, 451 mg).

Step B: 6-(Tetrahydroquinoline-1-sulfonyl)-2H-pyridazin-3-one. A mixture of 3-methoxy-6-(tetrahydroquinoline-1-sulfonyl)-pyridazine (1.14 mmol, 112 mg), conc. HCl (2 mL), and dioxane (5 mL) was heated at 100° C. for two hours. The reaction mixture was cooled and evaporated to dryness. Water (10 mL) was added to the residue and extracted with EtOAc. The EtOAc extract was washed with water, collected, dried, filtered and the filtrate was evaporated to a residue, which was crystallized from ether to yield 6-(tetrahydroquinoline-1-sulfonyl)-2H-pyridazin-3-one (33%, 11 mg); mp 200° C.

### EXAMPLE 48

### 6-(2.3-Tetrahydro-indole-1-sulfonyl)2H-pyridazin-3-one

The title compound of Example 48 was prepared from 2,3-tetrahydro-indole in a manner analogous to Example 47. (44%); mp >220° C.

### EXAMPLE 49

### 6-(5-Chloro-3-methyl-benzofuran-2-sulfinyl)-2H-pyridazin-3-one

A mixture of 6-(5-chloro-3-methyl-benzofuran-2-sulfenyl)-2H-pyridazin-3-one (prepared according to the method of Example 2, Step B) (5.0 g, 17.0 mmol), peracetic acid (1.9 g, 25.0 mmol) and acetic acid (20 mL) was stirred at room temperature for two hours. The reaction mixture was quenched with ice-cold water (30 mL) and the precipitated solid was filtered. The solid residue was washed with water (2×10 mL) and then air-dried to obtain the title compound of Example 49 (3.55 g, 73%); mp 234° C.-236° C.

### EXAMPLE 50

### 6-(5-Chloro-3-methyl-benzofuran-2-sulfonyl)-2H-pyridazin-3-one, sodium salt

To a solution of 6-(5-chloro-3-methyl-benzofuran-2-sulfonyl)-2H-pyridazin-3-one (2 mmol, 696 mg) in acetone (200 mL) was added powdered sodium hydroxide (2 mmol, 80 mg). After a precipitate formed in the clear solution, the solid was filtered off to obtain the title compound of Example 50 (90%, 628 mg). mp >260° C.

### EXAMPLE 51

### 2-Methyl-5-trifluoromethyl benzofuran

The title compound was prepared by following the procedure described in Tetrahedron Letters, 1988, 29, 4687-4690.

### EXAMPLE 52

### 4-Fluorophenyl-benzofuran

To an ice-cold solution of 3-coumaranone (10 mmol, 1.34 g) in ether (20 mL) was added 4-fluoro-phenyl magnesium bromide (2 Molar in ether, 20 mmol, 10 mL) and the reaction stirred for 3.5 hours. The reaction was quenched with H2O (10 mL), the pH was adjusted to 7 with sufficient 10% HCl and extracted with ether (3×10 mL). The ether extract was collected, dried, filtered, and evaporated to dryness. The residue was purified by silica gel chromatography (eluent: hexanes) to obtain 4-fluorophenyl-benzofuran.

### EXAMPLE 53

### 6-(3-Trifluoromethyl-benzenesulfonyl)-2H-pyridazin-3-one.

A mixture of 3,6-dichloropyridazine (4.44 g), 3-trifluoromethylphenyl sulfinic acid sodium salt (6.93 g), isopropanol (30 mL), and water (1 mL) was prepared and refluxed for 18 hours. The reaction mixture was then cooled, diluted with water (100 mL) and the precipitated solid was collected. The solid was triturated with n-propanol and the solid was collected to obtain the title compound (25%, 2.3 g).

### EXAMPLE 54

### 6-(2-Fluoro-benzenesulfonyl)-2H-pyridazin-3-one

Step A: 3-(2-Fluoro-phenylsulfanyl)-6-methoxy-pyridazine. To a clear solution of 4-fluorothiophenol (2.56 g) in DMF (10 mL) was added 3-chloro-6-methoxy-pyridazine (3.18 g) and stirred at room temperature for 1 hour. The reaction mixture was quenched with water (30 mL) and extracted with ethyl acetate (50 mL). The ethyl acetate layer was collected, washed with water (2×20 mL) and the organic portion was collected, dried over anhydrous sodium sulfate, filtered and the filtrate was evaporated to obtain crude 3-(2-fluoro-phenylsulfanyl)-6-methoxy-pyridazine (85%, 4.0 g, mp, 58-62° C.; mass spectrum M⁺, 236).

Step B : 3-(2-Fluoro-benzenesulfonyl)-6-methoxy-pyridazine. A mixture of 3-(2-fluoro-phenylsulfanyl)-6-methoxy-pyridazine (500 mg), m-chloroperbenzoic acid (MCPBA) (1.04 g) and methylene dichloride (10 mL) was prepared and stirred at room temperature for two hours. The reaction mixture was diluted with methylene dichloride and the methylene dichloride layer was washed with saturated sodium bicarbonate (10 mL) and then with water (2×20 mL). The methylene dichloride layer was collected, dried over anhydrous sodium sulfate, filtered and the filtrate was evaporated to dryness. The residue was purified by silica gel chromatography (3:1 ethyl acetate/hexane as eluent) to obtain 3-(2-fluoro-benzenesulfonyl)-6-methoxy-pyridazine as a white solid (51%, 290 mg; NMR, 4.19 (s, 3H), 7.13 (d, 1H), 7.21 (d, 1H), 8.13 (m, 4H).

Step C: 6-(2-Fluoro-benzenesulfonyl)-2H-pyridazin-3-one. A mixture of 3-(2-fluoro-benzenesulfonyl)-6-methoxy-pyridazine (200 mg) and concentrated hydrochloric acid (2 mL) was prepared and refluxed for one hour. The reaction mixture was cooled and diluted with water (20 mL). Sufficient 40% aqueous sodium hydroxide was then added to adjust the pH of the mixture to 3 and the mixture was extracted with ethyl acetate (2×20 mL). The ethyl acetate extract portions were collected and combined, dried over anhydrous sodium sulfate and filtered. The filtrate was evaporated to obtain the title compound as a white solid (45%, 80 mg), mp, 173-176° C.; NMR, 7.06 (d, 1H), 7.23 (m, 1H), 7.3 (m, 1H), 7.89 (d, 1H), 8.02 (m, 2H) and 11.66 (s, 1H).

### EXAMPLE 55

### 6-(4-Bromo-2-fluoro-benzenesulfonyl)-2H-pyridazin-3-one

Step A: 3-(4-Bromo-2-fluoro-phenylsulfanyl)-6-methoxy-pyridazine. A mixture of 2-fluoro-4-bromothiophenol (300 mg), 2,6-dichloro-pyridazine (149 mg), potassium carbonate (400 mg) and acetone (6 mL) was prepared and refluxed for two hours. The acetone from the mixture was evaporated and the resulting residue was dissolved in a solution of methanol (3 mL) and sodium metal (166 mg). The resulting solution was refluxed for 1 hour. Evaporation of methanol afforded 3-(4-bromo-2-fluoro-phenylsulfanyl)-6-methoxy-pyridazine, which was not isolated but was immediately used in Step 2.

Step B: 3-(4-Bromo-2-fluoro-benzenesulfonyl)-6-methoxy-pyridazine. The product of Step A (400 mg) was dissolved in chloroform (10 mL) and m-chloroperbenzoic acid (MCPBA) (770 mg) was added to the resulting solution. The reaction mixture was stirred overnight at room temperature. The solvent was evaporated and the resulting residue was purified by silica gel chromatography (90% hexane/10% ethyl acetate as eluent) to obtain the title compound (264 mg, 60%): mass spectrum, M⁺, 346.

Step C: 6-(4-Bromo-2-fluoro-benzenesulfonyl)-2H-pyridazin-3-one. A mixture of 3-(4-bromo-2-fluoro-benzenesulfonyl)-6-methoxy-pyridazine (260 mg), dioxane (5 mL), and concentrated hydrochloric acid (1 mL) was prepared and refluxed for two hours. The reaction mixture was then evaporated to dryness. The resulting residue was triturated with water and the precipitated solid was collected and air-dried to obtain the title compound (90%, 225 mg); mp, >220° C.; NMR 7.05 (d, 1H), 7.7 (d, 1H), 7.9 (m, 3H), 13.8 (s, 1H).

### EXAMPLE 56

### 6-(3-Chloro-benzenesulfonyl)-2H-pyridazin-3-one

Step A: 3-(3-Chloro-phenylsulfanyl)-6-methoxy-pyridazine. Sodium metal (218 mg) was dissolved in methanol (10 mL). 3-Chlorothiophenol was added and stirred for one hour at room temperature. The excess methanol was evaporated and to the dry residue was added toluene (20 mL) and 3-chloro-6-methoxypyridazine (1.1 g). The reaction mixture was refluxed for four hours, cooled to room temperature and then poured into water (30 mL). The pH of the solution was first adjusted to 10 with 20% potassium hydroxide and extracted with ethyl acetate (2×20 mL). The aqueous layer from the extraction was collected. The aqueous portion was acidified to pH 3 with concentrated hydrochloric acid and then extracted with ethyl acetate (3×10 mL). The ethyl acetate extract was evaporated and the residue was purified by silica gel chromatography to afford 3-(3-chloro-phenylsulfanyl)-6-methoxy-pyridazine (M⁺, 253).

Step B: 3-(3-Chloro-benzenesulfonyl)-6-methoxy-pyridazine. A mixture of 3-(3-chloro-phenylsulfanyl)-6-methoxy-pyridazine (529 mg), m-chloroperbenzoic acid (MCPBA) (760 mg) and chloroform (20 mL) was prepared and stirred at room temperature for two hours. The reaction mixture was diluted with 5% sodium thiosulfate (20 mL) followed by water (30 mL). The chloroform layer was collected, dried over anhydrous sodium sulfate, filtered and the dried chloroform portion was evaporated to dryness. The resulting solid residue was purified by silica gel chromatography (3:1 hexane/ethyl acetate as eluent) to obtain 3-(3-chloro-benzenesulfonyl)-6-methoxy-pyridazine (29%, 173 mg); mass spectrum, M⁺, 285.

Step C: 6-(3-Chloro-benzenesulfonyl)-2H-pyridazin-3-one. A mixture of 3-(3-chloro-benzenesulfonyl)-6-methoxy-pyridazine (148 mg), dioxane (2 mL) and concentrated hydrochloric acid (0.5 mL) was prepared and refluxed for 30 minutes. The reaction mixture was then evaporated to dryness and the residue was extracted with ethyl acetate (2×10 mL). The ethyl acetate mixture was collected, dried over anhydrous sodium sulfate, filtered and the filtrate was evaporated to dryness to afford 6-(3-chloro-benzenesulfonyl)-2H-pyridazin-3-one as white solid (38%, 61 mg); mp, 222-223° C.: NMR, 7.11 (d, 1H), 7.74 (t, 1H), 7.86-8.04 (m, 4H), 13.86 (s, 1H).

Examples 56A to 56N were prepared from the appropriate starting materials in a manner analogous to the method of Example 56.

| Example | Compound | MP ° C. |
|---|---|---|
| 56A | 6-(4-Fluoro-benzenesulfonyl)-2H-pyridazin-3-one | >225 |
| 56B | 6-(4-Trifluoromethyl-benzenesulfonyl)- | >220 |
| | 2H-pyridazin-3-one | |
| 56C | 6-(2-Bromo-benzenesulfonyl)-2H-pyridazin-3-one | 210-213 |
| 56D | 6-(3,4-Dichloro-benzenesulfonyl)-2H-pyridazin- | 166-168 |
| | 3-one | |
| 56E | 6-(4-Methoxy-benzenesulfonyl)-2H-pyridazin- | 111-113 |
| | 3-one | |
| 56F | 6-(2-Chloro-4-fluoro-benzenesulfonyl)-2H- | 205-208 |
| | pyridazin-3-one | |
| 56G | 6-(4-Chloro-benzenesulfonyl)-2H-pyridazin-3-one | >220 |
| 56H | 6-(2-Chloro-benzenesulfonyl)-2H-pyridazin-3-one | 220-222 |
| 56I | 6-(3-Bromo-benzenesulfonyl)-2H-pyridazin-3-one | >220 |
| 56K | 6-(4-Bromo-2-fluoro-phenylmethanesulfonyl)- | >220 |
| | 2H-pyridazin-3-one | |
| 56L | 6-(2,6-Dichloro-phenylmethanesulfonyl)-2H- | 219-220 |
| | pyridazin-3-one | |
| 56M | 6-(3-Chloro-5-methyl-benzenesulfonyl)-2H- | >250 |
| | pyridazin-3-one | |
| 56N | 6-(2-Chloro-4,6-difluoro-benzenesulfonyl)-2H- | >250 |
| | pyridazin-3-one | |

### Example 57

### 6-(2,4-Dichloro-benzenesulfonyl)-2H-pyridazin-3-one

Step A: 6-(2,4-Dichloro-phenylsulfanyl)-2H-pyridazin-3-one. Potassium t-butoxide (1.1 g) was added to a solution of 2,4-dichlorothiophenol (1.8 g) in N,N-dimethylformamide (DMF) (5 mL). The mixture was stirred at room temperature for 10 minutes and then 6-chloro-2H-pyridazin-3-one (1.31 g) was added. The reaction mixture was stirred at 100° C. for five hours. The mixture was then cooled to room temperature, poured into water (20 mL) and 20% potassium hydroxide (5 mL) was added. The resulting dark solution was extracted with ethyl acetate (2×10 mL). The aqueous layer was collected and the pH was adjusted to 3 with concentrated hydrochloric acid. The solution was then extracted with ethyl acetate (3×10 mL). The ethyl acetate layer was collected, dried over anhydrous sodium sulfate, filtered and evaporated to obtain a crude product, which was purified by silica gel chromatography (1:1 ethyl acetate/hexane as eluent) to afford 6-(2,4-dichloro-phenylsulfanyl)-2H-pyridazin-3-one (418 mg, 15%); NMR 6.88 (d, 1H), 7.10 (d, 1H), 7.24 (dd, 1H), 7.48 (d, 1H), 7.52 (d, 1H).

Step B: 6-(2.4-Dichloro-benzenesulfonyl)-2H-pyridazin-3-one. A mixture of 6-(2,4-dichloro-phenylsulfanyl)-2H-pyridazin-3-one (418 mg), peracetic acid (3.2 mL) and acetic acid (3.2 mL) was prepared and stirred for 2.5 hours at 80° C. The reaction mixture was then cooled to room temperature and poured into water (50 mL). The resulting white solid was collected and dried to obtain the title product, 6-(2,4-dichloro-benzenesulfonyl)-2H-pyridazin-3-one, (37%, 173 mg); mp, 202-203° C.; NMR 7.15 (d, 1H), 7.81 (dd, 1H), 8.03 (m, 2H), 8.25 (d, 1H), 13.88 (s, 1H).

Examples 57A to 57I were prepared from the appropriate starting materials in a manner analogous to the method of Example 57.

| Example | Compound | MP ° C. |
|---|---|---|
| 57A | 6-(2-Chloro-benzenesulfonyl)-2H-pyridazin-3-one | 220-222 |
| 57B | 6-(2,4-Difluoro-benzenesulfonyl)-2H-pyridazin- | 186-188 |
| | 3-one | |
| 57C | 6-(Naphthalene-1-sulfonyl)-2H-pyridazin-3-one | 225-226 |
| 57D | 6-(2,4-Dichloro-benzenesulfonyl)-2H-pyridazin- | 202-203 |
| | 3-one | |
| 57E | 6-(2-Fluoro-benzenesulfonyl)-2H-pyridazin-3-one | 189-191 |
| 57F | 6-(2,3-Dichloro-benzenesulfonyl)-2H-pyridazin- | 224-225 |
| | 3-one | |
| 57G | 6-(2,5-Dichloro-benzenesulfonyl)-2H-pyridazin- | 229-232 |
| | 3-one | |
| 57H | 6-(2,6-Dichloro-benzenesulfonyl)-2H-pyridazin- | 118-120 |
| | 3-one | |
| 57I | 6-(2,3-Difluoro-benzenesulfonyl)-2H-pyridazin- | >225 |
| | 3-one | |

### EXAMPLE 58

### 6-(2-Hydroxy-benzenesulfonyl)-2H-pyridazin-3-one

A mixture of 6-(2-methoxy-benzenesulfonyl)-2H-pyridazin-3-one (100 mg) and aluminum tri-bromide (2 g) was prepared and heated at 100° C. for two hours. The reaction mixture was cooled and water (10 mL) was added. The mixture was then extracted with chloroform. The organic extract was washed with water (2×10 mL), dried over anhydrous sodium sulfate and evaporated. The resulting residue was triturated with isopropyl ether and the resulting solid was collected by filtration to afford the title compound (61%, 58 mg), ¹HNMR (CDCl₃, 300 MHz), δ 7.0 (m, 3H), 7.6 (m, 2H), 7.8 (d, 1H).

### EXAMPLE 59

### 3-(2-Chloro-benzenesulfonyl)-6-methoxy-pyridazine, N-oxide

A mixture of 3-(2-chloro-phenylsulfanyl)-6-methoxy-pyridazine, m-chloroperbenzoic acid (MCPBA) (4.0 g), and chloroform (30 mL) was prepared and refluxed for 30 hours. Mass spectrum analysis of an aliquot of the reaction sample showed complete conversion to the desired sulfone-N-oxide (M+, 301). The reaction was cooled, washed successively with sodium sulfite (10% solution, 20 mL), sodium carbonate (10% solution, 20 mL), and water (2×20 mL). The chloroform layer was collected, dried over anhydrous sodium sulfate, filtered and the filtrate was evaporated to obtain a crude solid. The crude solid was purified by silica gel chromatography (1:1 ethyl acetate/hexane as eluent) to afford the title compound (38%, 425 mg); mp, 148-153° C.; (38%, 425 mg); NMR δ 4.01 (s, 3H), 6.80 (d, 1H), 7.42 (m, 1H), 7.57 (m, 2H), 8.38 (d, 1H), 8.46 (m, 1H).

### EXAMPLE 60

### 3-(2-Chloro-4-fluoro-benzenesulfonyl)-6-methoxy-pyridazine, N-oxide

The title compound was prepared according to a procedure analgous to that of Example 59 using 3-(2-chloro-4-fluoro-phenylsulfanyl)-6-methoxy-pyridazine as the starting compound. (60%); mp, 159-161° C.; NMR δ 4.01 (s, 3H), 6.80 (d, 1H), 7.15 (dd, 1H), 7.25 (dd, 1H), 8.37 (d, 1H), 8.49 (m, 1H).

### EXAMPLE 61

### 3-(2-Chloro-benzenesulfonyl)-6-methoxy-pyridazine

A mixture of 3-(2-chloro-benzenesulfonyl)-6-methoxy-pyridazine, N-oxide, N-oxide from Example 59 (317 mg) and triethyphosphite (3 mL) was heated to 100° C. for four hours. The reaction mixture was cooled to room temperature, poured into water (20 mL), and extracted with ethyl acetate (2×10 mL). The organic extract was evaporated to dryness and the crude product was purified by silica gel chromatography (1:1 ethyl acetate/hexane as eluent). (48%, 143 mg); NMR δ 4.19 (s, 3H), 7.19 (d, 1H), 7.43 (dd, 2H), 7.58 (m, 2H), 8.27 (d, 1H), 8.44 (dd, 2H).

### EXAMPLE 62

### 3-(2-Chloro-4-fluoro-benzenesulfonyl)-6-methoxy-pyridazine

The title compound was prepared according to procedure of Example 61 starting from 3-(2-chloro-4-fluoro-benzenesulfonyl)-6-methoxy-pyridazine, N-oxide. (48%); mp, 84-87° C.

### EXAMPLE 63

### 6-Methoxy-pyridazine-3-sulfonyl fluoride

Step A: 6-Methoxy-pyridazine-3-thiol. A mixture of 3-chloro-6-methoxy-pyridazine (100 g), thiourea (105 g) and ethyl methyl ketone (1.8 L) was prepared and refluxed for three hours. The reaction mixture was then cooled and the supernatant was poured into water and extracted with 1 M sodium hydroxide (4×100 mL). The sodium hydroxide solution was washed with ethyl acetate (2×50 mL) and the aqueous extract was acidified with sufficient concentrated hydrochloric acid to lower the pH to 5. The resulting yellow solid was collected and air dried to afford the title compound (24%, 23 g); mp, 198-200° C.

Step B: 6-Methoxy-pyridazine-3-sulfonyl fluoride. A mixture of 6-methoxy-pyridazine-3-thiol (7.1 g), methanol (100 mL), water (100 mL), and potassium hydrogen fluoride (39 g) was prepared and stirred at −10° C. for 30 minutes. Chlorine gas was bubbled into the mixture at a rate to ensure that the temperature did not exceed −10° C. The whitish-yellow reaction mixture was then poured into ice-cold water (50 mL) and the resulting white solid was filtered and air dried to afford the title compound (74%, 7.1 g); mp, 87-88° C.

### EXAMPLE 64

### 6-Oxo-1,6-dihydro-pyridazine-3-sulfonic acid methyl-phenyl-amide

Step A: 6-Methoxy-pyridazine-3-sulfonic acid methyl-phenyl-amide. A mixture was prepared of 6-methoxy-pyridazine-3-sulfonyl fluoride from Example 63 (1.62 mmol, 312 mg) and N-methyl aniline (24.3 mmol, 0.26 mL) and heated at 100° C. for 12 hours. The mixture was then cooled. The resulting solid residue was purified by silica gel chromatography to isolate the title compound (53%, 240 mg); M⁺, 279.

Step B: 6-Oxo-1.6-dihydro-pyridazine-3-sulfonic acid methyl-phenyl-amide. A mixture of 6-methoxy-pyridazine-3-sulfonic acid methyl-phenyl-amide (239 mg), dioxane (4 mL) and concentrated hydrochloric acid (1 mL) was prepared and refluxed for one hour. The mixture was then evaporated to dryness. The resulting solid was triturated with water and the solid was collected to afford the title compound (75%, 171 mg); mp, 157-158° C.

### EXAMPLE 65

### 6-Oxo-1,6-dihydro-pyridazine-3-sulfonic acid isopropyl-phenyl-amide

The title compound was prepared according to a procedure analogous to that of Example 64 for 6-oxo-1,6-dihydro-pyridazine-3-sulfonic acid methyl-phenyl-amide, substituting N-isopropylaniline for N-methyl aniline in step 3, (20%); mp, 190-191° C.

### EXAMPLE 66

### 6-Oxo-1,6-dihydro-pyridazine-3-sulfonic acid (3,4-dichloro-phenyl)-methyl-amide

The title compound was prepared according to a procedure analogous to that of Example 64 for 6-oxo-1,6-dihydro-pyridazine-3-sulfonic acid methyl-phenyl-amide, substituting N-methyl-3,4-dichloroaniline for N-methylaniline (28%); mp, 207-208° C.

### EXAMPLE 67

### 6-(4-Fluoro-phenylsulfanyl)-2H-pyridazin-3-one

A mixture of 3-(4-fluoro-phenylsulfanyl)-6-methoxy-pyridazine (250 mg), prepared by a procedure analogous to step A of Example 54, and concentrated hydrochloric acid was prepared and refluxed for 30 minutes. The mixture was then evaporated to dryness. The resulting residue was purified by silica gel chromatography (ethyl acetate as eluent) to afford the title compound (65%, 152 mg); mp, 99-101° C.

### EXAMPLE 68

### 6-(Biphenyl-4-sulfonyl)-2H-pyridazin-3-one

Step A: 3-(Biphenyl-4-sulfonyl)-6-methoxy-pyridazine. A mixture of 4-fluoro-benzene boronic acid (157 mg) 3-(4-fluoro-benzensulfonyl)-6-methoxy-pyridizine (247 mg), potassium carbonate (207 mg), Pd[P(Ph)₃]₄ (87 mg), toluene (4 mL), ethanol (2 mL) and water (1.5 mL) was prepared and refluxed for four hours. The mixture was cooled and water was added (10 mL). The mixture was then filtered and the resulting filtrate was extracted with ethyl acetate (20 mL). The ethyl acetate extract was washed with water and the ethyl acetate portion was collected and dried with anhydrous sodium sulfate and filtered. The filtrate was collected and evaporated to dryness to afford the title product of step A. NMR δ 4.17 (s, 3H), 7.13 (m, 3H), 7.54 (m, 2H), 7.70 (m, 2H), 8.17 (m, 3H).

Step B: 6-(Biphenyl-4-sulfonyl)-2H-pyridazin-3-one. The product of step A was treated with concentrated hydrochloric acid according to step C of Example 54 to obtain the title compound. Mp. 219-220° C.

### EXAMPLE 69

### 6-Benzyloxy-pyridazine-3-sulfonyl fluoride

Step A: 3-Benzyloxy-6-chloro-pyridazine. Sodium metal (3.1 g) was added to benzyl alcohol (75 mL) and gently warmed to 50° C. for 30 minutes until all the sodium metal dissolved. A solution of 3,6-dichloropyridazine (135 mmol) in benzyl alcohol (75 mL) was added. The reaction mixture was kept at 100° C. for 24 hours. Excess benzyl alcohol was evaporated and the residue was extracted with ethyl acetate (3×100 mL) and the ethyl acetate extract was washed with water. The resulting ethyl acetate layer was collected, dried, filtered, and the filtrate was evaporated to afford the title compound (90%, 26.7 g); mp, 77-78° C.

Step 2: 6-Benzyloxy-pyridazine-3-thiol. A mixture of 3-benzyloxy-6-chloro-pyridazine (4 g), thiourea (2.8 g) and ethyl methyl ketone (75 mL) was prepared and refluxed overnight. Excess ethyl methyl ketone was evaporated and the resulting residue was extracted with 2M sodium hydroxide (25 mL). The sodium hydroxide solution was then washed with ethyl acetate (2×30 mL). The aqueous layer was collected and sufficient concentrated hydrochloric acid was added to bring the pH to 5. The resulting solution was extracted with ethyl acetate (2×30 mL). The ethyl acetate extract was collected, dried, filtered, and the filtrate was evaporated to afford the title compound (15%, 605 mg); mp, 155-157° C.

Step 3: 6-Benzyloxy-pyridazine-3-sulfonyl fluoride. A mixture of 6-benzyloxy-pyridazine-3-thiol (510 mg), methanol (10 mL), water (10 mL), and potassium hydrogen fluoride (1.83 g) was prepared and stirred at −10° C. for 30 minutes. Chlorine gas was bubbled into the mixture at a rate to ensure that the temperature not exceed −10° C. The resulting whitish-yellow reaction mixture was poured into ice cold water (50 mL) and the resulting white solid was filtered and air-dried to afford the title compound. (Yield 89%, 560 mg); mp, 85-86° C.

### EXAMPLE 70

### 6-[2-(4-Chloro-phenyl)-2-oxo-ethanesulfonyl]-2H-pyridazin-3-one

Step A: 1-(4-Chloro-phenyl)-2-(6-methoxy-pyridazin-3-ylsulfanyl)-ethanone. A mixture of 2-mercapto-6-methoxy-pyridazine (1.42 g), 4-chloro-α-bromo acetophenone (10 mmol, 2.33 g), potassium carbonate (2.76 g), and dimethyl formamide (15 mL) was stirred at room temperature for one hour. The reaction mixture was filtered, the residue was washed with ethyl acetate (2×20 mL) and the combined filtrate was washed with water (2×20 mL). The ethyl acetate layer was collected, dried, filtered and the filtrate was evaporated to dryness to afford the title compound of step A (96%, 2.85 g); mass spectrum, m⁺295.

Step B: 1-(4-Chloro-phenyl)-2-(6-methoxy-pyridazine-3-sulfonyl)-ethanone. A mixture of the compound from step A, (8.5 mmol, 2.3 g), MCPBA (25 mmol, 5.8 g), and methylene chloride (160 mL) was stirred at room temperature for 40 min. To the reaction mixture was added a saturated solution of sodium bi-carbonate (400 mL) and the methylene chloride layer was collected, dried, filtered and the filtrate was evaporated to afford the title compound of step B as a white solid (79%, 2.2 g); mp, 153-156° C.

Step C: 6-[2-(4-Chloro-phenyl)-2-oxo-ethanesulfonyl]-2H-pyridazin-3-one. The compound from step B was transformed to the title compound, through acid hydrolysis, according to Step C, of Example 54; (79%); mp, >240° C.

### EXAMPLE 71

### 6-[2-(4-Chloro-phenyl)-2-hydroxy-ethanesulfonyl]-2H-pyridazin-3-one

A suspension was prepared of 6-[2-(4-chloro-phenyl)-2-oxo-ethanesulfonyl]-2H-pyridazin-3-one (1.0 mmol, 312 mg) prepared according to Example 70 in methanol (10 mL). Sodium borohydride (1.5 mmol, 55 mg) was added to the suspension at room temperature and stirred for 1 hour. The reaction mixture was evaporated and the residue was triturated with 10% hydrochloric acid (5 mL). The resulting white precipitate was filtered and air-dried to afford the title compound (69%, 218 mg); mp, 178-179° C.

### EXAMPLE 72

### Protocol for Determination of Aldose Reductase Inhibition

Test compound (TC) solutions were prepared by dissolving TC in 20 μl 20% dimethylsulfoxide (DMSO) and diluting with 100 mM potassium phosphate buffer, pH 7.0, to various TC concentrations, typically ranging from 5 mM to 1 μM. A “zero TC” solution was prepared that started with only 20 μF DMSO (no TC). The assay for aldose reductase activity was performed in a 96-well plate. Initiation of the reaction (with substrate) was preceded by a 10 minute pre-incubation at 24° C. of 200 μl 100 mM potassium phosphate buffer, pH 7.0, containing 125 pM NADPH and 12.5 nM human recombinant Aldose Reductase (Wako Chemicals, Inc., #547-00581) with 25 μl TC solution. The reaction was initiated by the addition of 25 μl 20 mM D-glyceraldehyde (Sigma, St. Louis). The rate of decrease in OD₃₄₀ was monitored for 15 minutes at 24° C. in a 340 ATTC Plate Reader (SLT Lab Instruments, Austria). Inhibition by TC was measured as the percentage decrease in the rate of NADPH oxidation as compared to a non-TC containing sample.

1. A pharmaceutical composition comprising a first compound selected from:
a compound of formula I 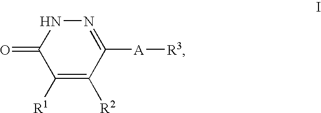
and a compound of formula II 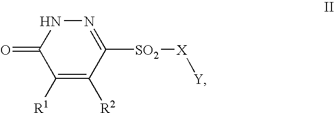
or a prodrug of said first compound, or a pharmaceutically acceptable salt of said first compound or said prodrug,
wherein:
A is S, SO or SO₂;
R¹ and R² are each independently hydrogen or methyl;
R³ is Het¹, —CHR⁴Het¹ or NR⁶R⁷;
R⁴ is hydrogen or (C₁-C₃)alkyl;
R⁶ is (C₁-C₆)alkyl, aryl or Het²;
R⁷ is Het³;
Het¹ is pyridyl, pyrimidyl, pyrazinyl, pyridazinyl, quinolyl, isoquinolyl, quinazolyl, quinoxalyl, phthalazinyl, cinnolinyl, naphthyridinyl, pteridinyl, pyrazinopyrazinyl, pyrazinopyridazinyl, pyrimidopyridazinyl, pyrimidopyrimidyl, pyridopyrimidyl, pyridopyrazinyl, pyridopyridazinyl, pyrrolyl, furanyl, thienyl, imidazolyl, oxazolyl, thiazolyl, pyrazolyl, isoxazolyl, isothiazolyl, triazolyl, oxadiazolyl, thiadiazolyl, tetrazolyl, indolyl, benzofuranyl, benzothienyl, benzimidazolyl, benzoxazolyl, benzothiazolyl, indazolyl, benzisoxazolyl, benzisothiazolyl, pyrrolopyridyl, furopyridyl, thienopyridyl, imidazolopyridyl, oxazolopyridyl, thiazolopyridyl, pyrazolopyridyl, isoxazolopyridyl, isothiazolopyridyl, pyrrolopyrimidyl, furopyrimidyl, thienopyrimidyl, imidazolopyrimidyl, oxazolopyrimidyl, thiazolopyrimidyl, pyrazolopyrimidyl, isoxazolopyrimidyl, isothiazolopyrimidyl, pyrrolopyrazinyl, furopyrazinyl, thienopyrazinyl, imidazolopyrazinyl, oxazolopyrazinyl, thiazolopyrazinyl, pyrazolopyrazinyl, isoxazolopyrazinyl, isothiazolopyrazinyl, pyrrolopyridazinyl, furopyridazinyl, thienopyridazinyl, imidazolopyridazinyl, oxazolopyridazinyl, thiazolopyridazinyl, pyrazolopyridazinyl, isoxazolopyridazinyl or isothiazolopyridazinyl; Het¹ is independently optionally substituted with up to a total of four substituents independently selected from R⁸, R⁹, R¹⁰ and R¹¹; wherein R⁸, R⁹, R¹⁰ and R¹¹ are each taken separately and are each independently halo, formyl, (C₁-C₆)alkoxycarbonyl, (C₁-C₆)alkylenyloxycarbonyl, (C₁-C₄)alkoxy-(C₁-C₄)alkyl, C(OH)R¹²R¹³, (C₁-C₄)alkylcarbonylamido, (C₃-C₇)cycloalkylcarbonylamido, phenylcarbonylamido, phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, (C₁-C₄)alkylsulfenyl, (C₁-C₄)alkylsulfonyl, (C₃-C₇)cycloalkyl, (C₁-C₄)alkyl optionally substituted with up to three fluoro or (C₁-C₄)alkoxy optionally substituted with up to five fluoro; said phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, in the definition of R⁸, R⁹, R¹⁰ and R¹¹ are optionally substituted with up to three substituents independently selected from hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said imidazolyl, oxazolyl, isoxazolyl, thiazolyl and pyrazolyl in the definition of R⁸, R⁹, R₁₀ and R₁₁ are optionally substituted with up to two substituents independently selected from hydroxy, halo, C₁-C₄)alkyl, hydroxy-(C₁-C₄)alkyl, (C₁-C₄)alkoxy-(C₁-C₄)alkyl, C₁-C₄)alkyl-phenyl optionally substituted in the phenyl portion with one Cl, Br, OMe, Me or SO₂-phenyl wherein said SO₂-phenyl is optionally substituted in the phenyl portion with one Cl, Br, OMe, Me, (C₁-C₄)alkyl optionally substituted with up to five fluoro, or (C₁-C₄)alkoxy optionally substituted with up to three fluoro;
R¹² and R¹³ are each independently hydrogen or (C₁-C₄)alkyl;
Het² and Het³ are each independently imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy; Het² and Het³ are each independently optionally substituted with up to a total of four substituents independently selected from R¹⁴, R¹⁵, R¹⁶ and R¹⁷, wherein R¹⁴, R¹⁵, R¹⁶ and R¹⁷ are each taken separately and are each independently halo, formyl, (C₁-C₆)alkoxycarbonyl, (C₁-C₆)alkylenyloxycarbonyl, (C₁-C₄)alkoxy-(C₁-C₄)alkyl, C(OH)R¹⁸R¹⁹, (C₁-C₄)alkylcarbonylamido, (C₃-C₇)cycloalkylcarbonylamido, phenylcarbonylamido, phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, (C₁-C₄)alkylsulfenyl, (C₁-C₄)alkylsulfonyl, (C₃-C₇)cycloalkyl, (C₁-C₄)alkyl optionally substituted with up to three fluoro or (C₁-C₄)alkoxy optionally substituted with up to five fluoro; said phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, in the definition of R¹⁴, R¹⁵, R¹⁶ and R¹⁷ are optionally substituted with up to three substituents independently selected from hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said imidazolyl, oxazolyl, isoxazolyl, thiazolyl and pyrazolyl in the definition of R¹⁴, R¹⁵, R¹⁶ and R¹⁷ are optionally substituted with up to two substituents independently selected from hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to three fluoro; and R¹⁸ and R¹⁹ are each independently hydrogen or (C₁-C₄)alkyl;
X and Y together are CH₂—CH(OH)—Ar or CH₂—C(O)—Ar, or
X is a covalent bond, NR²⁰ or CHR², wherein, R²⁰ is (C₁-C₃)alkyl or a phenyl that is optionally substituted with one or more substituents selected from OH, F, Cl, Br, I, CN, CF₃, (C₁-C₆)alkyl, O—(C₁-C₆)alkyl, S(O)ₙ—(C₁-C₆)alkyl and SO₂—NR²²R²³, and R²¹ is hydrogen or methyl, and
Y is a phenyl or naphthyl ring optionally substituted with one or more substituents selected from Ar, OH, F, Cl, Br, I, CN, CF₃, (C₁-C₆)alkyl, O—(C₁-C₆)alkyl, S(O)ₙ—(C₁-C₆)alkyl and SO₂—NR²²R²³;
Ar is a phenyl or naphthyl ring optionally substituted with one or more substituents selected from F, Cl, Br, I, CN, CF₃, (C₁-C₆)alkyl, O—(C₁-C₆)alkyl, S(O)ₙ—(C₁-C₆)alkyl and SO₂—NR²²R²³;
n is independently for each occurrence 0, 1 or 2;
R²² is independently for each occurrence H, (C₁-C₆)alkyl, phenyl or naphthyl; and
R²³ is independently for each occurrence (C₁-C₆)alkyl, phenyl or naphthyl, provided that when R³ is NR⁶R⁷, then A is SO₂; and a second compound that is a cyclooxygenase-2 inhibitor, a prodrug of said second compound or a pharmaceutically acceptable salt of said second compound or said prodrug.

2. A composition of claim 1 wherein said first compound is a compound of formula I, wherein A is SO₂; R¹ and R² are each hydrogen; R³ is Het¹, wherein Het¹ is 5H-furo-[3,2c]pyridin-4-one-2-yl, furano[2,3b]pyridin-2-yl, thieno[2,3b]pyridin-2-yl, indol-2-yl, indol-3-yl, benzofuran-2-yl, benzothien-2-yl, imidazo[1,2a]pyridin-3-yl, pyrrol-1-yl, imidazol-1-yl, indazol-1-yl, tetrahydroquinol-1-yl or tetrahydroindol-1-yl, wherein said Het¹ is optionally independently substituted with up to a total of two substituents each independently selected from fluoro, chloro, bromo, (C₁-C₆)alkyl, (C₁-C₆)alkoxy, trifluoromethyl, hydroxy, benzyl or phenyl; said benzyl and phenyl are each optionally independently substituted with up to three halo, (C₁-C₆)alkyl, (C₁-C₆)alkoxy, (C₁-C₆)alkylsulfonyl, (C₁-C₆)alkylsulfinyl, (C₁-C₆)alkylsulfenyl, trifluoromethyl or hydroxy, or a prodrug thereof or a pharmaceutically acceptable salt of said compound or prodrug.

3. A composition of claim 2 wherein Het¹ is indol-2-yl, benzofuran-2-yl, benzothiophen-2-yl, furano[2,3b]pyridin-2-yl, thieno[2,3b]pyridin-2-yl or imidazo[1,2a]pyridin-4-yl, wherein said Het¹ is optionally independently substituted with up to a total of two substituents independently selected from fluoro, chloro, bromo, (C₁-C₆)alkyl, (C₁-C₆)alkoxy, trifluoromethyl and phenyl; said phenyl being optionally substituted with up to two substituents independently selected from fluoro, chloro and (C₁-C₆)alkyl.

4. A composition of claim 1 wherein said first compound is selected from: 6-(3-trifluoromethyl-benzenesulfonyl)-2H-pyridazin-3-one;
6-(4-bromo-2-fluoro-benzenesulfonyl)-2H-pyridazin-3-one;
6-(4-trifluoromethyl-benzenesulfonyl)-2H-pyridazin-3-one;
6-(2-bromo-benzenesulfonyl)-2H-pyridazin-3-one;
6-(3,4-dichloro-benzenesulfonyl)-2H-pyridazin-3-one;
6-(4-methoxy-benzenesulfonyl)-2H-pyridazin-3-one;
6-(3-bromo-benzenesulfonyl)-2H-pyridazin-3-one;
6-(biphenyl-4-sulfonyl)-2H-pyridazin-3-one;
6-(4′-fluoro-biphenyl-4-sulfonyl)-2H-pyridazin-3-one;
6-(4′-trifluoromethyl-biphenyl-4-sulfonyl)-2H-pyridazin-3-one;
6-(3′,5′-bis-trifluoromethyl-biphenyl-4-sulfonyl)-2H-pyridazin-3-one;
6-(biphenyl-2-sulfonyl)-2H-pyridazin-3-one;
6-(4′-trifluoromethyl-biphenyl-2-sulfonyl)-2H-pyridazin-3-one;
6-(2-hydroxy-benzenesulfonyl)-2H-pyridazin-3-one;
6-(2-chloro-benzenesulfonyl)-2H-pyridazin-3-one;
6-(3-chloro-benzenesulfonyl)-2H-pyridazin-3-one;
6-(2,3-dichloro-benzenesulfonyl)-2H-pyridazin-3-one;
6-(2,5-dichloro-benzenesulfonyl)-2H-pyridazin-3-one;
6-(4-fluoro-benzenesulfonyl)-2H-pyridazin-3-one;
6-(4-chloro-benzenesulfonyl)-2H-pyridazin-3-one;
6-(2-fluoro-benzenesulfonyl)-2H-pyridazin-3-one;
6-(2,3-difluoro-benzenesulfonyl)-2H-pyridazin-3-one;
6-(2,4-dichloro-benzenesulfonyl)-2H-pyridazin-3-one;
6-(2,4-difluoro-benzenesulfonyl)-2H-pyridazin-3-one;
6-(2,6-dichloro-benzenesulfonyl)-2H-pyridazin-3-one;
6-(2-chloro-4-fluoro-benzenesulfonyl)-2H-pyridazin-3-one;
6-(2-bromo-4-fluoro-benzenesulfonyl)-2H-pyridazin-3-one; and
6-(naphthalene-1-sulfonyl)-2H-pyridazin-3-one,
or a prodrug thereof or a pharmaceutically acceptable salt of said compound or said prodrug.

5. A composition of claim 1 wherein said second compound is selected from celecoxib, rofecoxib and etoricoxib or a prodrug thereof or a pharmaceutically acceptable salt of said compound or said prodrug.

6. A pharmaceutical composition of claim 1 wherein said first compound is in an aldose reductase inhibiting amount.

7. A pharmaceutical composition of claim 1 wherein said second compound is present in a cyclooxygenase-2 inhibiting amount.

8. A pharmaceutical composition of claim 6 wherein said second compound is present in a cyclooxygenase-2 inhibiting amount.

9. A pharmaceutical composition of claim 1 further comprising a vehicle, diluent or carrier.

10-19 (canceled)

20. A therapeutic method comprising administering to a mammal in need of treatment or prevention of cardiac tissue ischemia a first compound selected from:
a compound of formula I 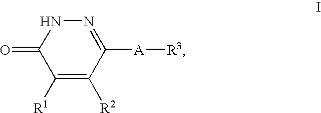
and a compound of formula II 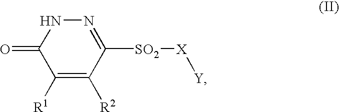
or a prodrug of said first compound, or a pharmaceutically acceptable salt of said first compound or said prodrug,
wherein:
A is S, SO or SO₂;
R¹ and R² are each independently hydrogen or methyl;
R³ is Het¹, —CHR⁴Het¹ or NR⁶R⁷;
R⁴ is hydrogen or (C₁-C₃)alkyl;
R⁶ is (C₁-C₆)alkyl, aryl or Het²;
R⁷ is Het³;
Het¹ is pyridyl, pyrimidyl, pyrazinyl, pyridazinyl, quinolyl, isoquinolyl, quinazolyl, quinoxalyl, phthalazinyl, cinnolinyl, naphthyridinyl, pteridinyl, pyrazinopyrazinyl, pyrazinopyridazinyl, pyrimidopyridazinyl, pyrimidopyrimidyl, pyridopyrimidyl, pyridopyrazinyl, pyridopyridazinyl, pyrrolyl, furanyl, thienyl, imidazolyl, oxazolyl, thiazolyl, pyrazolyl, isoxazolyl, isothiazolyl, triazolyl, oxadiazolyl, thiadiazolyl, tetrazolyl, indolyl, benzofuranyl, benzothienyl, benzimidazolyl, benzoxazolyl, benzothiazolyl, indazolyl, benzisoxazolyl, benzisothiazolyl, pyrrolopyridyl, furopyridyl, thienopyridyl, imidazolopyridyl, oxazolopyridyl, thiazolopyridyl, pyrazolopyridyl, isoxazolopyridyl, isothiazolopyridyl, pyrrolopyrimidyl, furopyrimidyl, thienopyrimidyl, imidazolopyrimidyl, oxazolopyrimidyl, thiazolopyrimidyl, pyrazolopyrimidyl, isoxazolopyrimidyl, isothiazolopyrimidyl, pyrrolopyrazinyl, furopyrazinyl, thienopyrazinyl, imidazolopyrazinyl, oxazolopyrazinyl, thiazolopyrazinyl, pyrazolopyrazinyl, isoxazolopyrazinyl, isothiazolopyrazinyl, pyrrolopyridazinyl, furopyridazinyl, thienopyridazinyl, imidazolopyridazinyl, oxazolopyridazinyl, thiazolopyridazinyl, pyrazolopyridazinyl, isoxazolopyridazinyl or isothiazolopyridazinyl; Het¹ is independently optionally substituted with up to a total of four substituents independently selected from R⁸, R⁹, R¹⁰ and R¹¹; wherein R⁸, R⁹, R¹⁰ and R¹¹ are each taken separately and are each independently halo, formyl, (C₁-C₆)alkoxycarbonyl, (C₁-C₆)alkylenyloxycarbonyl, (C₁-C₄)alkoxy-(C₁-C₄)alkyl, C(OH)R¹²R¹³ (C₁-C₄)alkylcarbonylamido, (C₃-C₇)cycloalkylcarbonylamido, phenylcarbonylamido, phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, (C₁-C₄)alkylsulfenyl, (C₁-C₄)alkylsulfonyl, (C₃-C₇)cycloalkyl, (C₁-C₄)alkyl optionally substituted with up to three fluoro or (C₁-C₄)alkoxy optionally substituted with up to five fluoro; said phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, in the definition of R⁸, R⁹, R¹⁰ and R¹¹ are optionally substituted with up to three substituents independently selected from hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said imidazolyl, oxazolyl, isoxazolyl, thiazolyl and pyrazolyl in the definition of R⁸, R⁹, R¹⁰ and R¹¹ are optionally substituted with up to two substituents independently selected from hydroxy, halo, C₁-C₄)alkyl, hydroxy-(C₁-C₄)alkyl, (C₁-C₄)alkoxy-(C₁-C₄)alkyl, C₁-C₄)alkyl-phenyl optionally substituted in the phenyl portion with one Cl, Br, OMe, Me or SO₂-phenyl wherein said SO₂-phenyl is optionally substituted in the phenyl portion with one Cl, Br, OMe, Me, (C₁-C₄)alkyl optionally substituted with up to five fluoro, or (C₁-C₄)alkoxy optionally substituted with up to three fluoro;
R¹² and R¹³ are each independently hydrogen or (C₁-C₄)alkyl;
Het² and Het³ are each independently imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy; Het² and Het³ are each independently optionally substituted with up to a total of four substituents independently selected from R¹⁴, R¹⁵, R¹⁶ and R¹⁷, wherein R¹⁴, R¹⁵, R¹⁶ and R¹⁷ are each taken separately and are each independently halo, formyl, (C₁-C₆)alkoxycarbonyl, (C₁-C₆)alkylenyloxycarbonyl, (C₁-C₄)alkoxy-(C₁-C₄)alkyl, C(OH)R¹⁸R¹⁹, (C₁-C₄)alkylcarbonylamido, (C₃-C₇)cycloalkylcarbonylamido, phenylcarbonylamido, phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, (C₁-C₄)alkylsulfenyl, (C₁-C₄)alkylsulfonyl, (C₃-C₇)cycloalkyl, (C₁-C₄)alkyl optionally substituted with up to three fluoro or (C₁-C₄)alkoxy optionally substituted with up to five fluoro; said phenyl, naphthyl, imidazolyl, pyridyl, triazolyl, benzimidazolyl, oxazolyl, isoxazolyl, thiazolyl, oxadiazolyl, thiadiazolyl, tetrazolyl, thienyl, benzothiazolyl, pyrrolyl, pyrazolyl, quinolyl, isoquinolyl, benzoxazolyl, pyridazinyl, pyridyloxy, pyridylsulfonyl, furanyl, phenoxy, thiophenoxy, in the definition of R¹⁴, R¹⁵, R¹⁶ and R¹⁷ are optionally substituted with up to three substituents independently selected from hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said imidazolyl, oxazolyl, isoxazolyl, thiazolyl and pyrazolyl in the definition of R¹⁴, R¹⁵, R¹⁶ and R¹⁷ are optionally substituted with up to two substituents independently selected from hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to three fluoro; and R¹⁸ and R¹⁹ are each independently hydrogen or (C₁-C₄)alkyl;
X and Y together are CH₂—CH(OH)—Ar or CH₂—C(O)—Ar, or
X is a covalent bond, NR²⁰ or CHR²¹, wherein, R²⁰ is (C₁-C₃)alkyl or a phenyl that is optionally substituted with one or more substituents selected from OH, F, Cl, Br, I, CN, CF₃, (C₁-C₆)alkyl, O—(C₁-C₆)alkyl, S(O)ₙ—(C₁-C₆)alkyl and SO₂—NR²²R²³, and R²¹ is hydrogen or methyl, and
Y is a phenyl or naphthyl ring optionally substituted with one or more substituents selected from Ar, OH, F, Cl, Br, I, CN, CF₃, (C₁-C₆)alkyl, O—(C₁-C₆)alkyl, S(O)ₙ—(C₁-C₆)alkyl and SO₂—NR²²R²³;
Ar is a phenyl or naphthyl ring optionally substituted with one or more substituents selected from F, Cl, Br, I, CN, CF₃, (C₁-C₆)alkyl, O—(C₁-C₆)alkyl, S(O)ₙ—(C₁-C₆)alkyl and SO₂—NR²²R²³;
n is independently for each occurrence 0, 1 or 2;
R²² is independently for each occurrence H, (C₁-C₆)alkyl, phenyl or naphthyl; and
R²³ is independently for each occurrence (C₁-C₆)alkyl, phenyl or naphthyl,
provided that when R³ is NR⁶R⁷, then A is SO₂, and a second compound that is a cyclooxygenase-2 inhibitor, a prodrug of said second compound or a pharmaceutically acceptable salt of said second compound or said prodrug.

21. A therapeutic method of claim 20 wherein said first compound is a compound of formula I, wherein A is SO₂; R¹ and R² are each hydrogen; R³ is Het¹, wherein Het¹ is 5H-furo-[3,2c]pyridin-4-one-2-yl, furano[2,3b]pyridin-2-yl, thieno[2,3b]pyridin-2-yl, indol-2-yl, indol-3-yl, benzofuran-2-yl, benzothien-2-yl, imidazo[1,2a]pyridin-3-yl, pyrrol-1-yl, imidazol-1-yl, indazol-1-yl, tetrahydroquinol-1-yl or tetrahydroindol-1-yl, wherein said Het¹ is optionally independently substituted with up to a total of two substituents each independently selected from fluoro, chloro, bromo, (C₁-C₆)alkyl, (C₁-C₆)alkoxy, trifluoromethyl, hydroxy, benzyl or phenyl; said benzyl and phenyl are each optionally independently substituted with up to three halo, (C₁-C₆)alkyl, (C₁-C₆)alkoxy, (C₁-C₆)alkylsulfonyl, (C₁-C₆)alkylsulfinyl, (C₁-C₆)alkylsulfenyl, trifluoromethyl or hydroxy, or a prodrug thereof or a pharmaceutically acceptable salt of said compound or prodrug.

22. A therapeutic method of claim 21 wherein Het¹ is indol-2-yl, benzofuran-2-yl, benzothiophen-2-yl, furano[2,3b]pyridin-2-yl, thieno[2,3b]pyridin-2-yl or imidazo[1,2a]pyridin-4-yl, wherein said Het¹ is optionally independently substituted with up to a total of two substituents independently selected from fluoro, chloro, bromo, (C₁-C₆)alkyl, (C₁-C₆)alkoxy, trifluoromethyl and phenyl; said phenyl being optionally substituted with up to two substituents independently selected from fluoro, chloro and (C₁-C₆)alkyl.

23. A therapeutic method of claim 20 wherein said first compound is selected from: 6-(3-trifluoromethyl-benzenesulfonyl)-2H-pyridazin-3-one;
6-(4-bromo-2-fluoro-benzenesulfonyl)-2H-pyridazin-3-one;
6-(4-trifluoromethyl-benzenesulfonyl)-2H-pyridazin-3-one;
6-(2-bromo-benzenesulfonyl)-2H-pyridazin-3-one;
6-(3,4-dichloro-benzenesulfonyl)-2H-pyridazin-3-one;
6-(4-methoxy-benzenesulfonyl)-2H-pyridazin-3-one;
6-(3-bromo-benzenesulfonyl)-2H-pyridazin-3-one;
6-(biphenyl-4-sulfonyl)-2H-pyridazin-3-one;
6-(4′-fluoro-biphenyl-4-sulfonyl)-2H-pyridazin-3-one;
6-(4′-trifluoromethyl-biphenyl-4-sulfonyl)-2H-pyridazin-3-one;
6-(3′,5′-bis-trifluoromethyl-biphenyl-4-sulfonyl)-2H-pyridazin-3-one;
6-(biphenyl-2-sulfonyl)-2H-pyridazin-3-one;
6-(4′-trifluoromethyl-biphenyl-2-sulfonyl)-2H-pyridazin-3-one;
6-(2-hydroxy-benzenesulfonyl)-2H-pyridazin-3-one;
6-(2-chloro-benzenesulfonyl)-2H-pyridazin-3-one;
6-(3-chloro-benzenesulfonyl)-2H-pyridazin-3-one;
6-(2,3-dichloro-benzenesulfonyl)-2H-pyridazin-3-one;
6-(2,5-dichloro-benzenesulfonyl)-2H-pyridazin-3-one;
6-(4-fluoro-benzenesulfonyl)-2H-pyridazin-3-one;
6-(4-chloro-benzenesulfonyl)-2H-pyridazin-3-one;
6-(2-fluoro-benzenesulfonyl)-2H-pyridazin-3-one;
6-(2,3-difluoro-benzenesulfonyl)-2H-pyridazin-3-one;
6-(2,4-dichloro-benzenesulfonyl)-2H-pyridazin-3-one;
6-(2,4-difluoro-benzenesulfonyl)-2H-pyridazin-3-one;
6-(2,6-dichloro-benzenesulfonyl)-2H-pyridazin-3-one;
6-(2-chloro-4-fluoro-benzenesulfonyl)-2H-pyridazin-3-one;
6-(2-bromo-4-fluoro-benzenesulfonyl)-2H-pyridazin-3-one; and
6-(naphthalene-1-sulfonyl)-2H-pyridazin-3-one, or a prodrug thereof or a pharmaceutically acceptable salt of said compound or said prodrug.

24. A therapeutic method of claim 20 wherein said second compound is selected from celecoxib, rofecoxib and etoricoxib or a prodrug thereof or a pharmaceutically acceptable salt of said compound or said prodrug.

25. A therapeutic method of claim 20 wherein said first compound is administered in an aldose reductase inhibiting amount.

26. A therapeutic method of claim 20 wherein said second compound is administered in a cyclooxygenase-2 inhibiting amount.

27. A therapeutic method of claim 25 wherein said second compound is administered in a cyclooxygenase-2 inhibiting amount.

28. A therapeutic method of claim 20 wherein said mammal is a human.
